SEQUENCE LISTING (36 sequences)

SEQ ID NO: 1 - RNA, 126 nt, synthetic construct
  misc_feature 1..126: note = Synthetic
  variation 31: note = n is a, c, g, or u
  variation 33: note = n is a, c, g, or u
  variation 74: note = n is a, c, g, or u
  variation 76: note = n is a, c, g, or u
  source 1..126: mol_type = other RNA
aaaaaaaaaaaaaaaaaaaaaaaaaaaaaangnaaaaaaaaaaaaaaaaaaaaaaaaaaaaaaaaaaaaaaaangnaaaaaaaaaaaaaaaaaaaaaaaaaaaaaaaaaaaaaaaaaaaaaaaaaa

SEQ ID NO: 2 - RNA, 123 nt, synthetic construct
  misc_feature 1..123: note = Synthetic
  variation 41: note = n is a, c, g, or u
  variation 43: note = n is a, c, g, or u
  source 1..123: mol_type = other RNA
aaaaaaaaaaaaaaaaaaaaaaaaaaaaaaaaaaaaaaaangnaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - RNA, 120 nt, synthetic construct
  misc_feature 1..120: note = Synthetic
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 128 nt, synthetic construct
  misc_feature 1..128: note = Synthetic
  variation 2: note = n is a, c, g, or u
  variation 33: note = n is a, c, g, or u
  variation 35: note = n is a, c, g, or u
  variation 76: note = n is a, c, g, or u
  variation 78: note = n is a, c, g, or u
  source 1..128: mol_type = other RNA
gnaaaaaaaaaaaaaaaaaaaaaaaaaaaaaangnaaaaaaaaaaaaaaaaaaaaaaaaaaaaaaaaaaaaaaaangnaaaaaaaaaaaaaaaaaaaaaaaaaaaaaaaaaaaaaaaaaaaaaaaaaa

SEQ ID NO: 5 - RNA, 115 nt, synthetic construct
  misc_feature 1..115: note = Synthetic
  variation 2: note = n is a, c, g, or u
  variation 33: note = n is a, c, g, or u
  variation 35: note = n is a, c, g, or u
  source 1..115: mol_type = other RNA
gnaaaaaaaaaaaaaaaaaaaaaaaaaaaaaangnaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - RNA, 122 nt, synthetic construct
  misc_feature 1..122: note = Synthetic
  variation 2: note = n is a, c, g, or u
  source 1..122: mol_type = other RNA
gnaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - RNA, 33 nt, synthetic construct
  misc_feature 1..33: note = Synthetic
  variation 1: note = n is a, c, g, or u
  variation 32: note = n is a, c, g, or u
  source 1..33: mol_type = other RNA
naaaaaaaaaaaaaaaaaaaaaaaaaaaaaang

SEQ ID NO: 8 - RNA, 43 nt, synthetic construct
  misc_feature 1..43: note = Synthetic
  variation 1: note = n is a, c, g, or u
  variation 42: note = n is a, c, g, or u
  source 1..43: mol_type = other RNA
naaaaaaaaaaaaaaaaaaaaaaaaaaaaaaaaaaaaaaaang

SEQ ID NO: 9 - RNA, 51 nt, synthetic construct
  misc_feature 1..51: note = Synthetic
  variation 1: note = n is a, c, g, or u
  source 1..51: mol_type = other RNA
naaaaaaaaaaaaaaaaaaaaaaaaaaaaaaaaaaaaaaaaaaaaaaaaaa

SEQ ID NO: 10 - RNA, 81 nt, synthetic construct
  misc_feature 1..81: note = Synthetic
  variation 1: note = n is a, c, g, or u
  source 1..81: mol_type = other RNA
naaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - RNA, 121 nt, synthetic construct
  misc_feature 1..121: note = Synthetic
  variation 1: note = n is a, c, g, or u
  source 1..121: mol_type = other RNA
naaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - RNA, 35 nt, synthetic construct
  misc_feature 1..35: note = Synthetic
  modified_base 1: mod_base = OTHER; note = 5' hydroxyl (-OH) group
  modified_base 35: mod_base = OTHER; note = 3'-phosphorylation
  source 1..35: mol_type = other RNA
aaaaaaaaaaaaaaaaaaaaaaaaaaaaaaaaatg

SEQ ID NO: 13 - RNA, 46 nt, synthetic construct
  misc_feature 1..46: note = Synthetic
  modified_base 1: mod_base = OTHER; note = 5' hydroxyl (-OH) group
  modified_base 46: mod_base = OTHER; note = 3'-phosphorylation
  source 1..46: mol_type = other RNA
cataaaaaaaaaaaaaaaaaaaaaaaaaaaaaaaaaaaaaaaaatg

SEQ ID NO: 14 - RNA, 53 nt, synthetic construct
  misc_feature 1..53: note = Synthetic
  modified_base 1: mod_base = OTHER; note = 5' hydroxyl (-OH) group
  modified_base 53: mod_base = OTHER; note = 3' hydroxyl (-OH) group
  source 1..53: mol_type = other RNA
cataaaaaaaaaaaaaaaaaaaaaaaaaaaaaaaaaaaaaaaaaaaaaaaaaa

SEQ ID NO: 15 - RNA, 47 nt, synthetic construct
  misc_feature 1..47: note = Synthetic
  modified_base 1: mod_base = OTHER; note = 5' hydroxyl (-OH) group
  modified_base 47: mod_base = OTHER; note = 3'-phosphorylation
  source 1..47: mol_type = other RNA
aaaaaaaaaaaaaaaaaaaaaaaaaaaaaaaaaaaaaaaaaaaaatg

SEQ ID NO: 16 - RNA, 83 nt, synthetic construct
  misc_feature 1..83: note = Synthetic
  modified_base 1: mod_base = OTHER; note = 5' hydroxyl (-OH) group
  modified_base 83: mod_base = OTHER; note = 3' hydroxyl (-OH) group
  source 1..83: mol_type = other RNA
cataaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 20 nt, synthetic construct
  misc_feature 1..20: note = Synthetic
  modified_base 20: mod_base = OTHER; note = 3' hydroxyl (-OH) group
  source 1..20: mol_type = other RNA
aaaaaaaaaaaaaaaaaaag

SEQ ID NO: 18 - RNA, 40 nt, synthetic construct
  misc_feature 1..40: note = Synthetic
  modified_base 40: mod_base = OTHER; note = 3' hydroxyl (-OH) group
  source 1..40: mol_type = other RNA
aaaaaaaaaaaaaaaaaaagaaaaaaaaaaaaaaaaaaag

SEQ ID NO: 19 - RNA, 60 nt, synthetic construct
  misc_feature 1..60: note = Synthetic
  modified_base 60: mod_base = OTHER; note = 3' hydroxyl (-OH) group
  source 1..60: mol_type = other RNA
aaaaaaaaaaaaaaaaaaagaaaaaaaaaaaaaaaaaaagaaaaaaaaaaaaaaaaaaag

SEQ ID NO: 20 - RNA, 80 nt, synthetic construct
  misc_feature 1..80: note = Synthetic
  modified_base 80: mod_base = OTHER; note = 3' hydroxyl (-OH) group
  source 1..80: mol_type = other RNA
aaaaaaaaaaaaaaaaaaagaaaaaaaaaaaaaaaaaaagaaaaaaaaaaaaaaaaaaagaaaaaaaaaaaaaaaaaaag

SEQ ID NO: 21 - RNA, 100 nt, synthetic construct
  misc_feature 1..100: note = Synthetic
  modified_base 100: mod_base = OTHER; note = 3' hydroxyl (-OH) group
  source 1..100: mol_type = other RNA
aaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaag

SEQ ID NO: 22 - RNA, 120 nt, synthetic construct
  misc_feature 1..120: note = Synthetic
  modified_base 120: mod_base = OTHER; note = 3' hydroxyl (-OH) group
  source 1..120: mol_type = other RNA
aaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaag

SEQ ID NO: 23 - RNA, 35 nt, synthetic construct
  misc_feature 1..35: note = Synthetic
  modified_base 35: mod_base = OTHER; note = 3' hydroxyl (-OH) group
  source 1..35: mol_type = other RNA
aaaaaaaaaaaaaaaaaaagaaaaaaaaaaaaaat

SEQ ID NO: 24 - RNA, 35 nt, synthetic construct; identical to SEQ ID NO: 23
  misc_feature 1..35: note = Synthetic
  modified_base 35: mod_base = OTHER; note = 3'-phosphorylation
  source 1..35: mol_type = other RNA

SEQ ID NO: 25 - RNA, 46 nt, synthetic construct
  misc_feature 1..46: note = Synthetic
  modified_base 46: mod_base = OTHER; note = 3' hydroxyl (-OH) group
  source 1..46: mol_type = other RNA
cataaaaaaaaaaaaaaaaaaagaaaaaaaaaaaaaataaaaaaaa

SEQ ID NO: 26 - RNA, 46 nt, synthetic construct; identical to SEQ ID NO: 25
  misc_feature 1..46: note = Synthetic
  modified_base 46: mod_base = OTHER; note = 3'-phosphorylation
  source 1..46: mol_type = other RNA

SEQ ID NO: 27 - RNA, 53 nt, synthetic construct
  misc_feature 1..53: note = Synthetic
  modified_base 53: mod_base = OTHER; note = 3' hydroxyl (-OH) group
  source 1..53: mol_type = other RNA
aaaaaaaaaaaaaaaaaaacaaaaaaaaaaaaaaaataaaaaaaaaaaaaaaa

SEQ ID NO: 28 - RNA, 53 nt, synthetic construct; identical to SEQ ID NO: 27
  misc_feature 1..53: note = Synthetic
  modified_base 53: mod_base = OTHER; note = 3'-phosphorylation
  source 1..53: mol_type = other RNA

SEQ ID NO: 29 - RNA, 47 nt, synthetic construct
  misc_feature 1..47: note = Synthetic
  modified_base 47: mod_base = OTHER; note = 3' hydroxyl (-OH) group
  source 1..47: mol_type = other RNA
aaaaaaaaaaaaaaaaaaagaaaaaaaaaaaaataaaaaaaaaaaaa

SEQ ID NO: 30 - RNA, 47 nt, synthetic construct; identical to SEQ ID NO: 29
  misc_feature 1..47: note = Synthetic
  modified_base 47: mod_base = OTHER; note = 3'-phosphorylation
  source 1..47: mol_type = other RNA

SEQ ID NO: 31 - RNA, 83 nt, synthetic construct
  misc_feature 1..83: note = Synthetic
  modified_base 83: mod_base = OTHER; note = 3' hydroxyl (-OH) group
  source 1..83: mol_type = other RNA
aaaaaaaaaaaaaaaaaaacaaaaaaaaaaaaaaaaaaataaaaaaaaaaaaaaaaaaagaaaaaaaaaaaaaaaaaagaaaa

SEQ ID NO: 32 - RNA, 35 nt, synthetic construct; identical to SEQ ID NO: 23
  misc_feature 1..35: note = Synthetic
  modified_base 35: mod_base = OTHER; note = N1-methyl-pseudouridine
  modified_base 35: mod_base = OTHER; note = 3'-phosphorylation
  source 1..35: mol_type = other RNA

SEQ ID NO: 33 - RNA, 46 nt, synthetic construct; identical to SEQ ID NO: 25
  misc_feature 1..46: note = Synthetic
  modified_base 3: mod_base = OTHER; note = N1-methyl-pseudouridine
  modified_base 38: mod_base = OTHER; note = N1-methyl-pseudouridine
  modified_base 46: mod_base = OTHER; note = 3'-phosphorylation
  source 1..46: mol_type = other RNA

SEQ ID NO: 34 - RNA, 53 nt, synthetic construct; identical to SEQ ID NO: 27
  misc_feature 1..53: note = Synthetic
  modified_base 37: mod_base = OTHER; note = N1-methyl-pseudouridine
  modified_base 53: mod_base = OTHER; note = 3' hydroxyl (-OH) group
  source 1..53: mol_type = other RNA

SEQ ID NO: 35 - RNA, 47 nt, synthetic construct; identical to SEQ ID NO: 29
  misc_feature 1..47: note = Synthetic
  modified_base 34: mod_base = OTHER; note = N1-methyl-pseudouridine
  modified_base 47: mod_base = OTHER; note = 3'-phosphorylation
  source 1..47: mol_type = other RNA

SEQ ID NO: 36 - RNA, 83 nt, synthetic construct; identical to SEQ ID NO: 31
  misc_feature 1..83: note = Synthetic
  modified_base 40: mod_base = OTHER; note = N1-methyl-pseudouridine
  modified_base 83: mod_base = OTHER; note = 3' hydroxyl (-OH) group
  source 1..83: mol_type = other RNA

(19) United States
(12) Patent Application Publication
(10) Pub. No.: US 2025/0002992 A1
(43) Pub. Date: Jan. 2, 2025

(54) METHODS OF ANALYZING AND CHARACTERIZING POLYADENYLATION ON MESSENGER RIBONUCLEIC ACID

(71) Applicant: Regeneron Pharmaceuticals, Inc., Tarrytown, NY (US)

(72) Inventors: Deanna Di Grandi, Goshen, NY (US); Yue Fu, Pleasantville, NY (US); Kathir Muthusamy, Cheshire, CT (US); Nisha Palackal, White Plains, NY (US); Daniel Dayeh, New York, NY (US)

(21) Appl. No.: 18/760,209

(22) Filed: Jul. 1, 2024

Related U.S. Application Data
(60) Provisional application No. 63/528,721, filed on Jul. 25, 2023.
(60) Provisional application No. 63/524,379, filed on Jun. 30, 2023.

(51) Int. Cl. C12Q 1/6869; C12Q 1/6806; C12Q 1/6811

(52) U.S. Cl. CPC C12Q 1/6869; C12Q 1/6806; C12Q 1/6811

(57) ABSTRACT

The inventions provide methods for characterizing messenger RNAs (mRNA) and polyadenylation on mRNAs. The disclosed inventions are developed for characterizing mRNA and poly(A) tail length on mRNA samples. These inventions can be used in release testing of mRNA drug substrate (DS) or drug product (DP), and can provide direct read-out of mRNA integrity and poly(A) tail length. The inventions further provide methods of analyzing the condition of mRNAs in samples. Characterized mRNA samples obtained by all of the inventive methods also are part of the inventions disclosed herein.

### CROSS REFERENCE TO RELATED APPLICATIONS

This application claims the benefit of and priority to U.S. Provisional Application No. 63/524,379 filed on Jun. 30, 2023, and U.S. Provisional Application No. 63/528,721 filed on Jul. 25, 2023. The above-referenced applications are incorporated herein by reference in their entirety.

### REFERENCE TO ELECTRONIC SEQUENCE LISTING

The application contains a sequence listing which has been submitted electronically in .xml format and is hereby incorporated by reference in its entirety. The .xml file, created on Jun. 26, 2024, is named “REGN 11474.xml” and is 75,949 bytes in size. The sequence listing contained in this .xml file is part of the specification and is hereby incorporated by reference herein in its entirety.

### TECHNICAL FIELD OF THE INVENTIONS

The present inventions provide for improved methods of analyzing and characterizing messenger ribonucleic acid (mRNA) and polyadenylation on mRNA. The inventions provide, among other things, improved approaches for evaluating, designing, producing and utilizing mRNA in the production of protein and other uses. Messenger RNA preparations provided by the inventive methods also are part of the inventions.

### BACKGROUND OF THE INVENTIONS

Messenger RNA has in the past been considered for medical therapies. Since the onset of the COVID-19 pandemic, in vitro transcribed (IVT) messenger ribonucleic acid (mRNA) has gained unprecedented attention and quickly proven itself to have the potential to become a new class of therapeutics for drug and vaccine development. Advances in addressing the challenges of IVT mRNA, particularly related to controlling translational efficacy and immunogenicity, will be needed for the successful translation of mRNA-based therapeutics from ideas to clinical approval.

Messenger RNA is normally considered to be the information shuttle between deoxyribonucleic acid (DNA) and translated proteins. Several components of mRNA are essential to its function, stability and translation efficiency, including a cap structure at a 5′ end, a 5′ untranslated region (UTR) and a 3′ UTR, and a polyadenylation (poly(A)) tail at a 3′ end. Notably, the presence of the 5′ cap and 3′ poly(A) tail are necessary for translation initiation and protein production; which generally occurs when the poly(A) tail interacts with a poly(A) binding protein, the 5′ cap and eukaryotic initiation factors (eIF).

Immediately after a gene in a eukaryotic cell is transcribed, the new mRNA molecule undergoes several modifications, which is known as mRNA processing. These modifications alter both ends of the primary mRNA transcript to produce a mature mRNA molecule. The processing of the 3′ end adds a poly(A) tail to the mRNA molecule. First, the 3′ end of the transcript is cleaved to free a 3′ hydroxyl. Then an enzyme called poly-A polymerase adds a chain of adenine nucleotides to the mRNA. This process, called polyadenylation, adds a poly(A) tail that is approximately 100 to 250 nucleotides (nt) in length. The poly(A) tail makes the RNA molecule more stable and prevents its degradation. Additionally, the poly(A) tail allows the mature messenger RNA molecule to be exported from the nucleus and translated into a protein by ribosomes in the cytoplasm. The presence of a poly(A) tail is a critical quality attribute for a messenger RNA vaccine. Messenger RNA molecules with and without a poly(A) tail (that is, tailless mRNA) can be separated and quantitated using ion pair reversed-phase high-performance liquid chromatography (IP-RP-HPLC, IP-RP-LC or IPRP-LC).

To mimic mRNA produced in cells, medicinal mRNA molecules experience deadenylation and terminate with a stretch of adenosines at their 3′ end and have various lengths. This deadenylation is the first step in mRNA decay as shorter poly(A) tails normally lead to a shorter half-life of mRNA molecules. The shortening of the 3′-poly(A) tail is the rate-limiting step in the degradation of most mRNAs and is important toward understanding the length impact of poly(A) tail.

For mRNA therapeutics or vaccines, the addition of poly(A) tails can be accomplished in three ways, including encoding poly(A) signal (PAS) into a template plasmid DNA, adding a poly(A) sequence to polymerase chain reaction (PCR) primers, or by adding a poly(A) sequence post IVT using polyadenylase. While each approach has both advantages and disadvantages, encoding PAS into a template plasmid DNA or adding a poly(A) sequence to PCR primers are of the most popular techniques used for mRNA-based therapeutics or vaccines.

Messenger RNA can degrade both in the coding regions and the poly(A) tail. Methods of analyzing and characterizing the entire mRNA and the poly(A) tail are needed.

To characterize the length and distribution of poly(A) tail in mRNA, current United States Pharmacopoeia (USP) guideline is to perform a RNA/DNA hybridization technique followed by site-specific cleavage of mRNA using RNAase H, reversed-phase high-performance liquid chromatography (RP-HPLC) separation, and various versions of a PCR-based polyadenylation test (poly(A) test or PAT) methods. Examples of PCR-based PAT methods may include rapid amplification of cDNA ends polyadenylation test (RACE-PAT), ligation-mediated polyadenylation test (LM-PAT), extension polyadenylation test (ePAT), splint-mediated polyadenylation test (sPAT), and high-resolution poly(A) tail assay (HIRE-PAT). While PCR-based PAT methods may provide information on the relative length of a poly(A) tail, they fail to provide a single nucleotide resolution nor the exact length of a poly(A) tail. Similarly, RP-HPLC separation only offers information regarding the percentage of mRNA containing poly(A) tail without further resolution into the length distribution of the tail. The PAT methods also utilize fluorescent-detection techniques to fluorescently tag poly(A) regions for amplification.

However, fluorescent-detection techniques, which implement a laser to achieve amplification readings, can be too harsh on the samples during poly(A) analysis such that they can affect the integrity of the sample over time; therefore, limiting the length of time the sample can be used for testing. Fluorescent-based dye techniques may include, for example, a direct label conjugated to the sample, or a fluorescent dye used in a separation buffer that will intercalate with the RNA once voltage is applied. Even more, fluorescent-based dye techniques also can produce an indirect readout (measurement) of the sample because only the dye that is able to complex to the RNA (instead of just measuring the RNA directly via its UV properties) is observed.

Among the PAT methods previously mentioned, the HIRE-PAT method provides similar resolution compared to the CGE method of the present inventions, whereas the other PAT methods provide much poorer resolution. However, the HIRE-PAT method implements fluorescent-detection techniques, specifically, fluorescent dye labeled PCR primers to add a fluorescence signal to be used with a fragment analyzer for detection of poly(A) regions for amplification. While the HIRE-PAT method can provide a single nucleotide resolution. However, because it utilizes fluorescence detection, it is not suited to be used for quantitation of each length due to at least the problems discussed above.

With the advancement of next generation sequencing (NGS), poly(A) tail length measurement has been implemented on different NGS platforms, including Illumina, PacBio and Nanopore. NGS methods can reveal minor sequence variation in mRNA and therefore depict the highest resolution (single nucleotide resolution). However, NGS can sometimes be complicated by the steps needed to perform the sequencing (for example, reverse-transcription from mRNA to DNA, and adding handles for identification, that is, adding identification in library preparation steps to align the reads for data analysis).

Additional complications include the bioinformatics support needed to interpret the sequencing data, and lack of quality control due to error-prone NGS platforms. Liquid Chromatography-Mass Spectrometry (LC-MS) has also been used to conduct a poly(A) tail length analysis. In particular, LC-MS offers single nucleotide resolution and a complex data analysis of poly(A) tail length distribution. However, like NGS, LC-MS is not ideal for release testing of mRNA drug substances (DS) due to the complexity of the LC-MS method, overall cost of execution, and instrument qualification.

In view of the need for improved characterization of mRNA and poly(A) tails on mRNA, the inventions provide improved methods for evaluating, designing, producing and utilizing mRNAs having poly(A) tails. The methods preferably achieve one or more of high resolution, low sample requirements, direct measurement capabilities, avoiding the need for reverse transcription and identification handles, and are amenable to quality control for good manufacturing practice (GMP) are provided by the inventions described herein.

### SUMMARY OF THE INVENTIONS

The inventions provide methods for characterizing a polyadenylated region on a messenger ribonucleic acid (mRNA) that further comprises a coding region are provided. For example, a method for characterizing a polyadenylation region on mRNA may include adjusting mRNA concentration in an mRNA sample based upon mRNA length. The method for characterizing a polyadenylation region on mRNA may include treating the mRNA sample with RNase T1 to remove the coding region and allow the polyadenylated region to remain. The method for characterizing a polyadenylation region on mRNA may include incubating the RNase T1 treated sample with oligo-dT beads. The method for characterizing a polyadenylation region on mRNA may include eluting the polyadenylated region bound to the oligo-dT beads. The method for characterizing a polyadenylation region on mRNA may include removing residual salts from the elution of the polyadenylated region bound to the oligo-dT beads. The method for characterizing a polyadenylation region on mRNA may include analyzing (using capillary gel electrophoresis (CGE), capillary electrophoresis (CE) or microchip capillary electrophoresis (MCE) the polyadenylated region after removing the residual salts from the elution of the polyadenylated region bound to the oligo-dT beads.

One or more of the following exemplary features may be included. The step of removing residual salts from the elution may include using nuclease free water. The step of removing residual salts from the elution may include using a Zeba spin, dialysis, tangential flow flotation, or CAT cartridges. The step of analyzing the polyadenylated region may include using a binding buffer comprising, for example, 20 mM Tris-HCl, a pH of 7.5, 2 mM EDTA, and 1000 mM LiCl. The step of analyzing the polyadenylated region may also include using a separation buffer comprising Tris-Borate-Urea. The step of analyzing the polyadenylated region may further include using a wash buffer comprising, for example, 10 mM Tris-HCl, a pH of 7.5, 1 mM EDTA, and 150 mM LiCl. The method for characterizing a polyadenylation region on mRNA may further include, prior to analyzing the polyadenylated region, mixing the mRNA sample with a population of mRNA ladders, wherein each mRNA ladder may comprise at least one poly(A) sequence member having a guanine nucleotide spacer, and wherein the mRNA ladder may have a nucleotide length of 12 to 250 nucleotides. Usually, the mRNA ladder will have a nucleotide length of 20 to 130 nucleotides, for example. The RNase T1 sample may comprise about 0.5 M salt to allow binding to the oligo-dT beads. The step of removing residual salts from the elution may facilitate CGE. The step of analyzing the polyadenylated region may utilize an ultraviolet (UV) detector for detecting the polyadenylated region. The UV detector may use a light at a wavelength of 250 to 260 nm. The UV detector preferably may use a light at a wavelength of 254 nm. The mRNA ladder may comprise a poly(A) sequence member having 10 to 25 adenine residues with a guanine nucleotide spacer, wherein the mRNA ladder may have a hydroxyl group at the 3′ end. The mRNA ladder may further comprise a plurality of poly(A) sequence members having 10 to 25 adenine residues with a guanine nucleotide spacer. The population of mRNA ladders may comprise (i) a first mRNA ladder comprising at least one poly(A) sequence member having 10 to 25 adenine residues with a guanine nucleotide spacer, wherein the first mRNA ladder may have a hydroxyl group at the 3′ end, and (ii) a second mRNA ladder may comprise at least one poly(A) sequence member having 10 to 25 adenine residues with a guanine nucleotide spacer, and wherein the second mRNA ladder may have a phosphate group at the 3′ end.

In another aspect, a characterized sample of a polyadenylated region of a messenger RNA (mRNA) that further comprises a coding region, may be produced. For example, producing the characterized sample may include adjusting mRNA concentration in an mRNA sample based upon mRNA length. Producing the characterized sample may include treating the mRNA sample with RNase T1 to remove the coding region and allow the polyadenylated region to remain. Producing the characterized sample may include incubating the RNase T1 treated sample with oligo-dT beads. Producing the characterized sample may include eluting the polyadenylated region bound to the oligo-dT beads. Producing the characterized sample may include removing residual salts from the elution of the polyadenylated region bound to the oligo-dT beads. Producing the characterized sample may include analyzing, using capillary gel electrophoresis (CGE), the polyadenylated region after removing the residual salts from the elution of the polyadenylated region bound to the oligo-dT beads.

One or more of the following exemplary features may be included. The step of removing residual salts from the elution may include using nuclease free water. The step of removing residual salts from the elution may include using a Zeba spin, dialysis, tangential flow flotation, or CAT cartridges. The step of analyzing the polyadenylated region may include using a binding buffer comprising, for example, 20 mM Tris-HCl, a pH of 7.5, 2 mM EDTA, and 1000 mM LiCl. The step of analyzing the polyadenylated region may also include using a separation buffer comprising Tris-Borate-Urea. The step of analyzing the polyadenylated region may further include using a wash buffer comprising, for example, 10 mM Tris-HCl, a pH of 7.5, 1 mM EDTA, and 150 mM LiCl. Producing the characterized sample may further include, prior to analyzing the polyadenylated region, mixing the mRNA sample with a population of mRNA ladders, wherein each mRNA ladder may comprise at least one poly(A) sequence member having a guanine nucleotide spacer, and wherein the mRNA ladder may have a nucleotide length of 12 to 250 nucleotides. Usually, the mRNA ladder will have a nucleotide length of 20 to 130 nucleotides, for example. The RNase T1 sample may comprise about 0.5 M salt to allow binding to the oligo-dT beads. The step of removing residual salts from the elution may facilitate CGE. The step of analyzing the polyadenylated region may utilize an ultraviolet (UV) detector for detecting the polyadenylated region. The UV detector may use a light at a wavelength of 250 nm to 260 nm. The UV detector may use a light at a wavelength of 254 nm.

The inventions further provide methods for characterizing polyadenylated regions on a messenger ribonucleic acids (mRNAs) that may further comprise coding regions, wherein the method may comprise the steps of: (a) adjusting mRNA concentration in an mRNA sample based upon mRNA length; (b) treating the mRNA sample with RNase T1 to remove the coding region and allow the polyadenylated region to remain; (c) incubating the RNase T1 treated sample with oligo-dT beads; (d) eluting the polyadenylated region bound to the oligo-dT beads; (c) removing residual salts from the elution of step (d); and (f) analyzing the polyadenylated region of step (e) using capillary gel electrophoresis (CGE). The methods may further comprise, prior to step (f), mixing the mRNA sample with a population of mRNA ladders, wherein each mRNA ladder may comprise at least one poly(A) sequence member having a guanine nucleotide spacer, wherein the mRNA ladder may have a nucleotide length of 12 to 250 nucleotides. Usually, the mRNA ladder will have a nucleotide length of 20 to 130 nucleotides, for example. The mRNA ladders may comprises a poly(A) sequence member having 10 to 25 adenine residues with a guanine nucleotide spacer, and wherein the mRNA ladder has a hydroxyl group at the 3′ end. The mRNA ladders may further comprise a plurality of poly(A) sequence members having 10 to 25 (or 18 to 22, or 19) adenine residues with a guanine nucleotide spacer. The population of mRNA ladders may comprise (i) a first mRNA ladder comprising a poly(A) sequence member having 10 to 25 (or 18 to 22, or 19) adenine residues with a guanine nucleotide spacer, and wherein the first mRNA ladder has a hydroxyl group at the 3′ end, and (ii) a second mRNA ladder comprising a poly(A) sequence member having 10 to 25 adenine residues with a guanine nucleotide spacer, and wherein the second mRNA ladder has a phosphate group at the 3′ end. The first mRNA ladder and the second mRNA ladder each further comprise at least one N1-methyl-pseudouridine nucleotide and/or a cytosine nucleotide.

The inventions also provide characterized samples of polyadenylated regions of messenger RNAs (mRNAs) that further comprise coding regions, wherein the characterized sample is produced according to the steps of: (a) adjusting mRNA concentration in an mRNA sample based upon mRNA length; (b) treating the mRNA sample with RNase T1 to remove the coding region and allow the polyadenylated region to remain; (c) incubating the RNase T1 treated sample with oligo-dT beads; (d) eluting the polyadenylated region bound to the oligo-dT beads; (c) removing residual salts from the elution of step (d); and (f) analyzing the polyadenylated region of step (c) using capillary gel electrophoresis (CGE). The characterized samples further comprise, prior to step (f), mixing the mRNA sample with a population of mRNA ladders, wherein each mRNA ladder comprises a poly(A) sequence member having a guanine nucleotide spacer, wherein the mRNA ladders may have a nucleotide length of 12 to 250 nucleotides. Usually, the mRNA ladders have a nucleotide length of 20 to 130 nucleotides, for example. Messenger RNA ladders may comprise a poly(A) sequence member having 10 to 25 (or 18 to 22, or 19) adenine nucleotides with a guanine nucleotide spacer, and wherein the mRNA ladder has a hydroxyl group at the 3′ end. The mRNA ladder may further comprise a plurality of poly(A) sequence members having 10 to 25 (or 18 to 22, or 19) adenine residues with a guanine nucleotide spacer. The population of mRNA ladders may comprise (i) a first mRNA ladder comprising at least one poly(A) sequence member having 10 to 25 (or 18 to 22, or 19) adenine residues with a guanine nucleotide spacer, and wherein the first mRNA ladder has a hydroxyl group at the 3′ end, and (ii) a second mRNA ladder comprising at least one poly(A) sequence member having 10 to 25 (or 18 to 22, or 19) adenine residues with a guanine nucleotide spacer, and wherein the second mRNA ladder has a phosphate group at the 3′ end. The first mRNA ladders and the second mRNA ladders each further comprise at least one N1-methyl-pseudouridine nucleotide.

The inventions also provide messenger RNA ladders that may comprise (i) at least one poly(A) sequence member having 10 to 25 (or 18 to 22, or 19) adenine nucleotides, (ii) at least one N1-methyl-pseudouridine nucleotide and (iii) at least one guanine nucleotide spacer, and wherein the mRNA ladder may have a hydroxyl group at the 3′ end. The messenger RNA ladders may further comprise at least one selected from the group consisting of a cytosine nucleotide and a uracil nucleotide. The messenger RNA ladder may further comprise a plurality of poly(A) sequence members having 10 to 25 adenine nucleotides with a guanine nucleotide spacer. The mRNA ladders can have a nucleotide length of 12 to 250 nucleotides, and usually 20 to 130 nucleotides.

The inventions further provide messenger RNA ladders that may comprise (i) at least one poly(A) sequence member having 10 to 25 (or 18 to 22, or 19) adenine nucleotides, (ii) at least one N1-methyl-pseudouridine nucleotide and (iii) at least one guanine nucleotide spacer, and wherein the mRNA ladder may have a phosphate group at the 3′ end. The messenger RNA ladders may further comprise at least one selected from the group consisting of a cytosine nucleotide and a uracil nucleotide. The messenger RNA ladder may further comprise a plurality of poly(A) sequence members having 10 to 25 adenine nucleotides with a guanine nucleotide spacer. The poly(A) sequence member may have 18 to 22 (or 19) adenine nucleotides. The mRNA ladders can have a nucleotide length of 12 to 250 nucleotides, and usually 20 to 130 nucleotides.

The inventions also provide populations of messenger RNA ladders, wherein the population may comprise (A) at least one messenger RNA ladder comprising (i) at least one poly(A) sequence member having 10 to 25 (or 18 to 22, or 19) adenine nucleotides, (ii) at least one N1-methyl-pseudouridine nucleotide and (iii) at least one guanine nucleotide spacer, and wherein the mRNA ladder may have a hydroxyl group at the 3′ end; and (B) at least one messenger RNA ladder comprising (i) at least one poly(A) sequence member having 10 to 25 (or 18 to 22, or 19) adenine residues, (ii) at least one N1-methyl-pseudouridine nucleotide and (iii) a guanine nucleotide spacer, and wherein the mRNA ladder may have a phosphate group at the 3′ end. The messenger RNA ladders may have at least one of (A) or (B) further comprise at least one selected from the group consisting of a cytosine nucleotide and a uracil nucleotide. The mRNA ladders may have a nucleotide length of 12 to 250 nucleotides. Usually, the mRNA ladders have a nucleotide length of 20 to 130 nucleotides, for example.

The inventions further provide methods for analyzing the condition of messenger ribonucleic acid molecules (mRNA) in a sample, wherein the method comprises the steps of: (a) analyzing the mRNA contained in the sample using capillary electrophoresis; and (b) comparing the migration time (and/or relative migration time) of the mRNA from step (a) to a migration time (an/or relative migration time) of intact mRNA. The migration time (and/or relative migration time) of intact mRNA may be determined using capillary electrophoresis, for example. The migration time (and/or relative migration time) and size of peaks may be compared. Migration time may slightly vary between electrophoresis runs, and thus runs may be repeated to yield a relative migration time to address these variations.

The samples may be subjected to degradation conditions, such as conditions selected from the group consisting of freeze/thaw cycles, unfrozen storage and elevated temperatures. For example, the degradation condition may be 25° C., 30° C., 35° C., 40° C., 45° C., 50° C., 55° C., 60° C., 65° C., 70° C., 75° C., 80° C., 85° C., 90° C., 95° C., 100° C. or more.

The data from the capillary electrophoresis may be used to calculate the half-life of the mRNA. Characterized sample obtained according to the methods also are provided.

Messenger RNA preparations provided by the inventive methods also are part of the inventions.

Further description of the inventions is provided below.

### BRIEF DESCRIPTION OF THE FIGURES

FIG. 1A (SEQ ID NOS: 1 to 3) is schematic diagram of three different poly(A) designs, including design A, design B, and design C. Design A and design B contain segmented poly(A) while design C contains continuous poly(A), wherein all three designs have a total of 120 adenosines. FIG. 1B (SEQ ID NOS: 4 to 11) is a schematic illustration of methods of workflow for poly(A) length and distribution analysis, particularly highlighting the resolution difference for 120-A poly(A) for AEX, IP-RP and CGE methodologies. The CGE methodology was determined to be the superior method, providing increased resolution for conducting poly(A) length and distribution analysis.

FIGS. 2A-2F depict a series of electropherograms respectively illustrating the length distribution of poly(A) analyzed under design A (see FIG. 1A) in combination with a CGE-UV methodology for three different mRNA constructs, including EGFP mRNA, Fly Luciferase mRNA, and Cas9 mRNA. From left to right are the measurements collected from EGFP mRNA, Fly Luciferase mRNA, and Cas9 mRNA, respectively. From top to bottom, the series of electropherograms depict isolated poly(A) tails for the three mRNA constructs with poly(A) 40 spike-in, wherein the length of each poly(A) segment can be read from the electropherogram and are labeled at the most abundant peak with an “*”, and isolated poly(A) tails for the three mRNA constructs without poly(A) 40 spike-in.

FIGS. 3A-3F depict a series of electropherograms respectively illustrating the length distribution of poly(A) analyzed under design B (see FIG. 1A) in combination with a CGE-UV methodology for three different mRNA constructs, including EGFP mRNA, Fly Luciferase mRNA, and Cas9 mRNA. From left to right are the measurements collected from EGFP mRNA, Fly Luciferase mRNA, and Cas9 mRNA, respectively. From top to bottom, the series of electropherograms depict isolated poly(A) tails for the three mRNA constructs with poly(A) 40 and poly(A) 80 spike-in, wherein the length of each poly(A) segment can be read from the electropherogram and are labeled at the most abundant peak with an “*”, and isolated poly(A) tails for the three mRNA constructs without the markers spike-in.

FIGS. 4A-4F depict a series of electropherograms respectively illustrating the length distribution of poly(A) analyzed under design C (see FIG. 1A) in combination with a CGE-UV methodology for three different mRNA constructs, including EGFP mRNA, Fly Luciferase mRNA, and Cas9 mRNA. From left to right are the measurements collected from EGFP mRNA, Fly Luciferase mRNA, and Cas9 mRNA, respectively. From top to bottom, the series of electropherograms depict isolated poly(A) tails for the three mRNA constructs with poly(A) 120 marker spike-in, wherein the length of each poly(A) segment can be read from the electropherogram and are labeled at the most abundant peak with an “*”, and isolated poly(A) tails for the three mRNA constructs without the markers spike-in.

FIGS. 5A-5C depict, for purposes of comparison to the CGE-UC methodology used in the present inventions, a series of electropherograms respectively illustrating the peak distribution of poly(A) analyzed under designs A, B, and C (see FIG. 1A) in combination with an AEX-LC methodology for Cas9 mRNA. From left to right, a n/n+1 resolution was achieved under design A, a 40-A poly(A) segment was achieved under design B, however, only partial n/n+1 resolution could be achieved for an 80-A poly(A) segment, and no n/n+1 resolution was achieved for a 120-A poly(A) segment under design C.

FIGS. 6A-6C depict, for purposes of comparison to the CGE-UC methodology used in the present inventions, a series of electropherograms respectively illustrating the peak distribution of poly(A) analyzed under designs A, B, and C (see FIG. 1A) in combination with an IP-RP-LC methodology for EGFP mRNA and Oxaloacetate (OAA) as immunoprecipitation (IP) reagent. From left to right, a n/n+1 resolution was achieved for a 40-A and 50-A poly(A) segment under design A, an 80-A poly(A) segment was achieved under design B, and a partial n/n+1 resolution was achieved for a 120-A poly(A) segment under design C. For the 40-A poly(A) segment in design A, the elution volume is closed to the initial noise region, highlighting the need to optimize LC gradient for specific poly(A) design.

FIGS. 7A-7C depict a series of electropherograms respectively illustrating the alignment of poly(A) marker(s) between 6-ladder mixture and spike-in for three different mRNA constructs. The isolated poly(A) from the three different mRNAs were analyzed using the set of injections (triplicates+6−ladder mixture+spike). The results achieved, from left to right, a 40-A alignment of EGFP mRNA, 40-A and 80-A alignment for Fly Luciferase mRNA, and 120-A alignment for EGFP mRNA, respectively.

FIGS. 8A-8C depict a series of electropherograms of isolated poly(A) analyzed under design A (see FIG. 1A) from three mRNA constructs and with poly(A) 40 and poly(A) 60 combined spike-in. From left to right are the measurements collected from EGFP mRNA, Fly Luciferase mRNA, and Cas9 mRNA, respectively. The results indicated that the poly(A) segments showed highly similar length distribution among the three mRNA constructs.

FIGS. 9A-9I depict a series of electropherograms of isolated poly(A) analyzed under design A (see FIG. 1A) using CGE triplicates of injections for three different mRNA constructs. From left to right are the measurements collected from EGFP mRNA, Fly Luciferase mRNA, and Cas9 mRNA, respectively.

FIGS. 10A-10I depict a series of electropherograms of isolated poly(A) analyzed under design B (see FIG. 1A) and using CGE triplicates of injections for three different mRNA constructs. From left to right are the measurements collected from EGFP mRNA, Fly Luciferase mRNA, and Cas9 mRNA, respectively.

FIGS. 11A-11I depict a series of electropherograms of isolated poly(A) analyzed under design C (see FIG. 1A) and using CGE triplicates of injections for three different mRNA constructs. From left to right are the measurements collected from EGFP mRNA, Fly Luciferase mRNA, and Cas9 mRNA, respectively.

FIG. 12 depicts an electropherogram of six poly(A) marker migration profiles using a CGE-UV methodology. A wide separation of markers was observed for all six lengths when using CGE.

FIGS. 13A-13B compare IPRP-LC and CE methods in terms of resolution. “RFU” used in FIG. 13B and throughout stands for “relative fluorescence units.” EGFP mRNA was tested.

FIG. 14 depicts absorbance vs time data showing separation of EGFP (enhanced green fluorescent protein mRNA), OVA (Ovalbumin mRNA) and FLuc (Fly Luciferase mRNA).

FIGS. 15A-15D depict RFU vs. time for EGFP, OVA and FLuc showing near-baseline resolution between main peaks.

FIGS. 16A-16D depict RFU vs time data for 6 replicate injections of FLuc.

FIG. 17 depicts percent (%) area at day 1 and day 2 using 6 replicate injections of FLuc, two different analysts and four types of analyzers.

FIG. 18 is a schematic depiction of an intact messenger RNA comprising a 5′ Cap region, a 5′ untranslated region (UTR), an open reading frame, a 3′ UTR and a poly(A) tail).

FIGS. 19A-19B depict RFU vs time data using EGFP (top) and Cas9 (CRISPR Associated Protein 9 mRNA) (bottom) at Day 0 (blue) and Day 6 at 40° C. (red) to distinguish between intact mRNA and degraded mRNA.

FIG. 20 depicts RFU vs. time data using EGFP (top) and Cas9 (bottom) using different types of analyzers.

FIG. 21 depicts purity (%) vs. time (days) for EGFP, OVA, FLuc and Cas9 showing mRNA degradation over time.

FIG. 22 depicts half-life data for EGFP, OVA, FLuc and Cas9 using five different analyzers.

FIG. 23 (SEQ ID NOS: 12 to 16) depicts the cleavage site of the RNase T1 enzyme after each guanidine residue (left side) and the resulting poly(A) sequence fragments for mRNA samples (from EGFP mRNA) with design A and design B treated with RNase T1, and the expected 3′ functional groups to include a 3′ phosphate group where there is a guanidine cleavage or a 3′ hydroxyl group where there is no guanidine cleavage (right side).

FIG. 24 (SEQ ID NOS: 17 to 31) depicts, for the purpose of comparison, an original set of size markers used to estimate the lengths of poly(A), each having a 3′ OH (upper). Round 1 shows an optimized set of size markers with length and base modifications and with either a 3′ P or a 3′ OH (lower).

FIG. 25 depicts a series of electropherograms of isolated poly(A) tail cleaved from an mRNA sample (from EGFP mRNA) with design A illustrating a comparison of different functional groups on the 3′ end of the spiked size marker (OH or P) and the effect on migration (for example, at 35 nt) and peak splitting (for example, at 46 nt). From top to bottom, the first panel shows the “neat” sample (isolated poly(A) tails without the marker spike-in) with different length distributions observed, and the size markers with (3′ OH, middle panel) and (3′ P, bottom panel) spiked-in to show the change in migration of the sequence caused by the 3′ functional groups. Peak splitting is evident at the second panel and third panel at 46 nt. At the right side of FIG. 25, two hypotheses are set forth to account for the observed peak splitting.

FIG. 26 depicts, for purpose of comparison, expected isolated poly(A) tail lengths under design A based on sequence, lengths determined using original ladder size markers (isolated poly(A) tails with the markers spiked-in) analyzed under design A, and optimized size markers selected for expected 3′ functional group based on the RNase T1 reaction (isolated poly(A) tails having a phosphate group or hydroxyl group on the 3′ end and with the marker spike-in) collected from the second and third panel of the electropherograms illustrated in FIG. 25.

FIG. 27 depicts, for purpose of comparison, expected isolated poly(A) tail lengths under design A based on encoded sequence to generate mRNA samples, lengths determined using original ladder size markers (isolated poly(A) tails with the markers spike-in) analyzed under design A, and optimized size markers selected for expected 3′ functional group based on the RNase T1 reaction (isolated poly(A) tails having a phosphate group or hydroxyl group on the 3′ end and with the marker spiked-in) collected from the second and third panel of the electropherograms illustrated in FIG. 25, and the lengths assessed by capillary electrophoresis mass spectrometry (CE-MS). The deconvoluted mass spectrum for the smallest fragment of design A is depicted in the graph above the table confirming the longest length to be 33 nt.

FIG. 28 (SEQ ID NOS: 17 to 36) depicts, for the purpose of comparison, an original set of size markers used to estimate the lengths of poly(A) tail, each having a 3′ OH (upper). Round 1 shows an optimized set of size markers with length and base modifications and with either a 3′ P or a 3′ OH (lower). Round 2 shows a further optimized set of size markers with length and base modification with an N1-methyl-pseudouridine (m1ψ) modification on all of the uridines and a 3′ phosphate group or a 3′ hydroxyl group as expected for the RNase T1 enzyme reaction.

FIG. 29 depicts two electropherograms of isolated poly(A) tail analyzed under design A respectively illustrating the use of the further optimized size markers named ‘Round 2’ and the disappearance of the observed peak spitting in FIG. 25. From top to bottom, the first panel shows the “neat” sample (isolated poly(A) tails without the markers spike-in) with different length distributions observed, and the second panel shows the optimized size marker with the N1-methyl-pseudouridine (m1ψ or N1M) modification on all of the uridines, optimized for the length and base composition, a 3′ phosphate (3′ P) for where there is a guanidine cleavage or 3′ hydroxyl (3′ OH) for where there is no guanidine cleavage.

### DETAILED DESCRIPTION OF THE INVENTION

### II. Definitions

It should be appreciated that this disclosure is not limited to the compositions and methods described herein as well as the experimental conditions described, as such may vary. It is also to be understood that the terminology used herein is for the purpose of describing certain aspects only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods, and materials similar or equivalent to those described herein can be used in the practice or testing of the present invention. All publications mentioned are incorporated herein by reference in their entirety.

All numerical limits and ranges set forth herein include all numbers or values thereabout or there between of the numbers of the range or limit. The ranges and limits described herein expressly denominate and set forth all whole numbers, integers, decimals and fractional values defined and encompassed by the range or limit within the context of usage. The ranges and limits described herein expressly denominate and set forth all whole numbers, integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (for example, “such as”) provided herein, is intended merely to better illuminate the inventions and does not pose a limitation on the scope of the inventions unless otherwise claimed. No language in the specification should be construed as indicating any non-claimed element as essential to the practice of the invention

The term “about” in the context of numerical values and ranges refers to values or ranges that approximate or are close to the recited values or ranges such that the inventions can perform, such as having a sought rate, amount, density, degree, increase, decrease, percentage, value or presence of a form, variant, temperature, or amount of time, as is apparent from the teachings contained herein. Thus, this term encompasses values beyond those simply resulting from systematic error. For example, “about” can signify values either above or below the stated value in a range of approx. +/−10% or more or less depending on the ability to perform.

“Anion-Exchange Chromatography (AEX)” refers to a form of ion exchange chromatography (IEX), which is used to separate molecules based on their net surface charge.

“Capillary Electrophoresis (CE)” refers to methods for separating biopolymers such as DNA, RNA, and proteins by size. In general, electrophoresis is a separation technique based on the migration of charged molecules in response to an electric field toward the electrode of opposite charge. CE typically utilizes polymer liquids in capillary tubes for separation, although can instead use polymer gels. See Capillary Gel Electrophoresis below.

“Capillary Gel Electrophoresis (CGE)” is a type of CE and refers to methods for separating biopolymers such as DNA, RNA, and proteins by molecular weight. In general, gel electrophoresis is a separation technique based on the migration of charged molecules in response to an electric field toward the electrode of opposite charge. Instead of liquid polymers, CGE utilizes polymer gels in capillary tubes for separation.

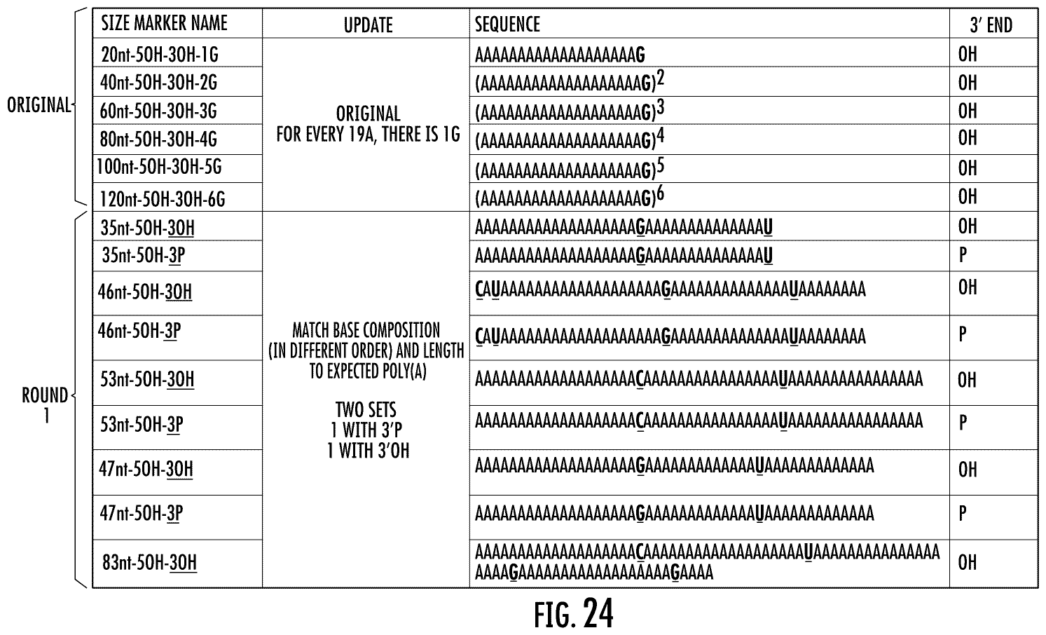

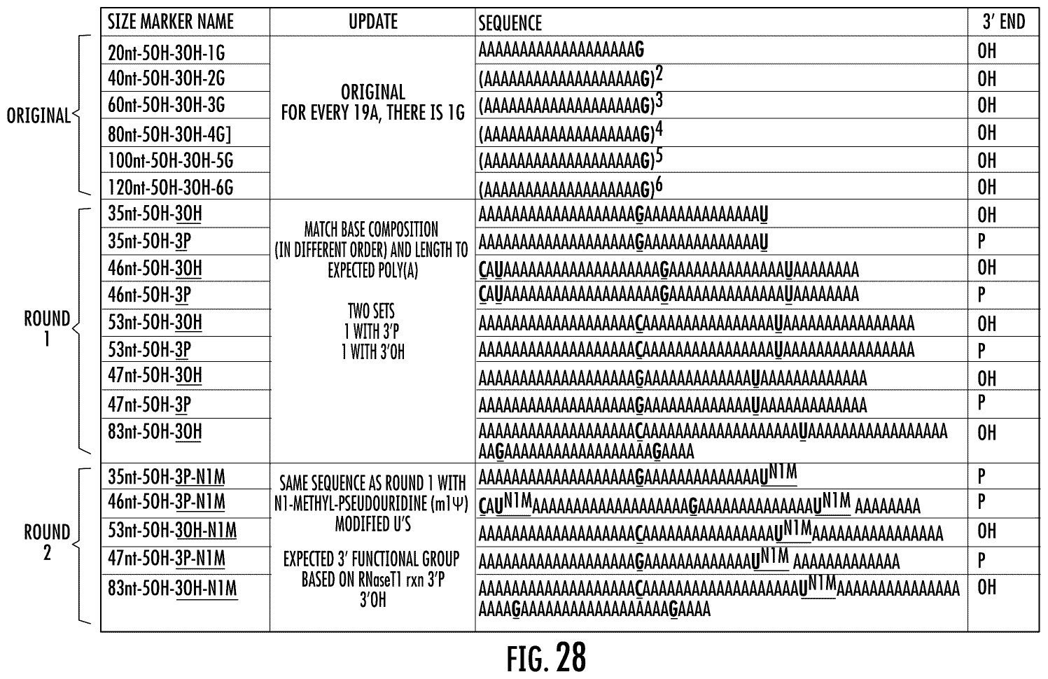

A “guanine nucleotide spacer” is a guanine nucleotide bound to 1 or 2 poly(A)) sequence members. See, for example, FIGS. 24 and 28.

“Microchip Capillary Electrophoresis (MCE) is a miniaturized type of CE.

“Ion-Pairing Reverse Phase Chromatography (IP-RP or IPRP)” refers to a separation technique by which a large amount of Ion-Pairing (IP) agent must be used to facilitate the separation of molecules that differ by only a single base pair.

“Heterogeneity” refers to the distribution of the poly(A) lengths. Theoretically, there should be a single homogenous population based on transcription from a defined DNA template. In practice, however, enzymes make errors that lead to a heterogeneous population comprised of a distribution of tail lengths. For example, poly(A) tail, although based on mRNA template sequence and should have a defined length, always exists as more than one length.

“Orthogonal method” or approach refers to different methods intended to measure the same attributes and are often necessary to provide independent confirmation of Critical Quality Attributes (CQAs). An orthogonal approach is used to monitor the same CQAs of a biotherapeutic formulation using different measurement principles to address biotherapeutic research questions.

A “poly(A) ladder” or “poly(A) marker” refers to a set of standards that are used for determining the approximate size of a protein or a nucleic acid fragment run on an electrophoresis gel. These standards contain pre-determined fragment (or protein) sizes and concentrations. Poly(A) ladders/markers each comprise at least one Poly(A) sequence and at least one guanine nucleotide space. Poly(A) ladders/markers also can comprise a N1-methyl-pseudouridine nucleotide (U^{NIM}) or a uracil nucleotide

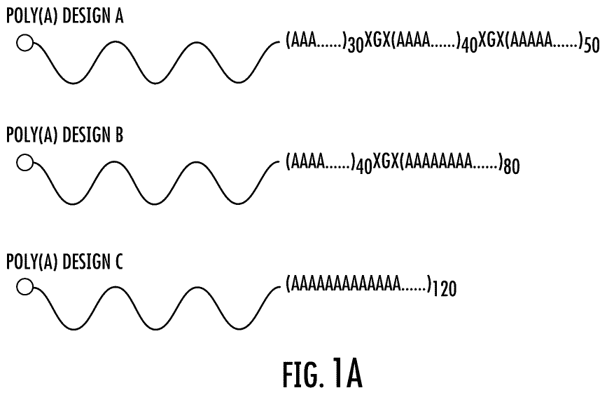

A “poly(A) sequence member” is a string of consecutive adenine nucleotide bound 5′ to 3′. Sec, for example, FIGS. 1A, 1B, 23, 24 and 28.

A “poly(A) tail” refers to the 3′ end of a mRNA molecule, and serves to protect the molecule from degradation and to facilitate protein translation. Poly(A) tails are typically up to about 250 nucleotides in length.

“Messenger RNA” (abbreviated mRNA) is a type of single-stranded ribonucleic acid involved in protein synthesis. Messenger RNA is made from a DNA template during the process of transcription.

“RNAase T1” refers to an endoribonuclease that specifically degrades single-stranded RNA at G residues. It cleaves the phosphodiester bond between the 3′-guanylic residue and the 5′-OH residue of adjacent nucleotides with the formation of corresponding intermediate 2′,3′-cyclic phosphate. The reaction products are 3′-GMP and oligonucleotides with a terminal 3′-GMP.

The phrase “spike-in” refers to adding a poly(A) marker/ladder directly into the poly(A) sample isolated from mRNA.

The phrase “combined spike-in” refers to more than one poly(A) markers being added to the poly(A) sample isolated from mRNA.

All numerical limits and ranges set forth herein include all numbers or values thereabout or there between of the numbers of the range or limit. The ranges and limits described herein expressly denominate and set forth all integers, decimals and fractional values defined and encompassed by the range or limit.

### II. Methods for Characterizing Poly(A) Tails on mRNA

The 3′ poly(A) tail is an important component of mRNA because it controls the stability and translation efficiency of the mRNA molecule. A poly(A) tail is required at the 3′ end of the mRNA molecule to protect the molecule from degradation and to facilitate successful protein translation.

Messenger RNA has emerged and established itself as one of the new modalities for drug and vaccine development. Notably, the presence of a poly(A) trail is a critical quality attribute for a therapeutic mRNA vaccine. For mRNA-based therapeutics or vaccines, and as part of quality control for mRNA drug substrates (DS) or drug products (DP), various analytical methods are used to characterize the critical quality attributes of the mRNA component, including the 5′ cap percent (%), purity, poly(A) tail and others. Such methods also require release testing depending upon the selection of the testing panel. However, current United States Pharmacopoeia (USP) guidelines only offer information regarding the percentage of mRNA containing poly(A) tail without further resolution into the length distribution of the tail; such as, for example, site-specific cleavage of mRNA using RNAase H followed by RP-HPLC separation. Other release methods for poly(A) tail involve even lower resolution methods; such as, for example, tris-borate-EDTA sodium dodecyl-sulfate polyacrylamide gel electrophoresis (TBE-SDS-PAGE).

In contrast, capillary gel electrophoresis (CGE) is a label-free, direct measurement method that, by optimization, can achieve single nucleotide resolution (high resolution) while necessitating only low sample requirements. Another advantage of CGE is that the instruments can be readily qualified and the cost of running CGE is much less compared to more complicated platforms. By adding a synthetic poly(A) ladder (also known as a poly(A) marker, and which allows for accurate sizing of poly(A) length beyond 100 nt), the CGE-UV method provide for a direct read-out of poly(A) length distribution using an electropherogram, instead of the flawed fluorescent-based dye techniques previously discussed.

The accuracy of the CGE-UV method in ascertaining a poly(A) length direct read-out was confirmed by testing the methodology against other orthogonal approaches, including capillary electrophoresis-mass spectrometry (CE-MS), as well as two types of LC methods: AEX-UV and IP-PR-UV. The comparison concluded that CGE was the recommended approach for achieving quality control-friendly and fit-for-purpose poly(A) tail length analysis.

The poly(A) ladder was designed to overcome the difficulties associated with chemical synthesis of consecutive stretches of adenosines. Technical challenges limit commercial vendors from synthesizing oligos with more than about 20 to 30 consecutive adenosines, making the production of longer Poly(A) ladders for mRNA research and development difficult. In the present inventions, marker design was achieved by introducing a guanosine into the poly-adenosine sequence at every 20th position. It was found that the use of another purine to replace adenine helps to minimize changes in molecular weight (Adenine, 135.13 Da; Guanine, 151.13 Da) compared to pyrimidines (Uracil, 112.09 Da; Cytosine, 111.10 Da) (Molecular weights reported from National Institute of Health National Library of Medicine, PubChem).

Assessing the heterogeneity of poly(A) tails in IVT mRNA is important from a clinical standpoint as mRNA is increasingly being produced for therapeutic purposes and because having a consistent and well-defined product is important to reproducible activity. Characterizing IVT mRNA through a CGE method for directly determining poly(A) tail length with single-nucleotide resolution that facilitates quality control and release testing, poly(A) tail length and distribution can be analyzed in less than 4 hours with most of time spent on poly(A) isolation from mRNA using an RNase T1 digestion treatment.

The CGE method of the present inventions characterizes IVT-produced mRNA of 1000 nucleotides (nt), 2000 nucleotides (nt), to 4500 nucleotides (nt), and having a poly(A) between 30 adenosines (30A) to 120 adenosines (120A). However, the upper limit of poly(A) that can be analyzed using the CGE method is approximately 140A to 150A; which is above regular mRNA in cells or recommended poly(A) length for optimal protein expression and longer than most of poly(A) tail design in mRNA used in therapeutics and vaccines.

The benefit of directly reading-out of poly(A) tail length without the need of extrapolation from standard curves or complex MS data analysis (compared with LC-MS analysis of poly(A)) makes the CGE method ideal for release testing of mRNA DS or DP. Additionally, the CGE method is poly(A) length independent and does not need to optimize gradient for specific poly(A) length (like in the case of IP-RP-LC method). As will be described in further detail, all three different poly(A) designs with poly(A) length from 30A to 120A can be analyzed using the same CGE method with n/n+1 resolution.

The present inventions provide a quality control friendly and fit-for-purpose poly(A) tail length analysis approach developed for determining mRNA poly(A) tail length and distribution. With the addition of the poly(A) ladder, the methods demonstrate a higher resolution comparable with liquid chromatography-mass spectrometry (LC-MS) based methods, and offer direct read-out of poly(A) tail length, both of which make it ideal for use in release testing of mRNA DS or DP.

Poly(A) tail length and distribution on mRNA was assessed using three different mRNA constructs, including enhanced green fluorescent protein (EGFP), Fly Luciferase, and Cas9, with lengths ranging from 1000 nt to 4500 nt. (See, Table 1). For each mRNA construct, three types of poly(A) were added to the 3′ end through a PCR-based approach (See, Table 1 and FIG. 1A (SEQ ID NOS: 1 to 3)).

TABLE 1
mRNA constructs and poly(A) designs

| mRNA | Approximate | Poly(A) | Poly(A) | Poly(A) |
|---|---|---|---|---|
| construct | Length (nt) | design A | design B | design C |
| EGFP | 1000 | 30A-40A-50A | 40A-80A | 120A |
| Fly Luciferase | 2000 | 30A-40A-50A | 40A-80A | 120A |
| Cas9 | 4500 | 30A-40A-50A | 40A-80A | 120A |

Poly(A) design A has three segments of poly(A), including a length of 30A, 40A and 50A, respectively. In poly(A) design A, each segment is separated by a spacer with at least one G nucleoside (Guanosine). Poly(A) design B contains two segments of poly(A), including one segment having a length of 40A and the second with a length of 80A. In poly(A) design B, the two segments are also separated by a short spacer; which has at least one G nucleoside. For poly(A) design C, a continuous poly(A) tail having a length of 120A was added. Notably, poly(A) design A and B, which contain segmented poly(A) tail (and have comparable translation efficiency with the secondary structure introduced by the G-containing spacers), could help reduce the formation of double-stranded RNA (dsRNA) impurities during IVT reaction.

Poly(A) markers/ladders can be of a size desired by the person skilled in the art. Typically, the markers will range from about 12 to 250 ribonucleotides in length, more typically about 20 to 150 ribonucleotides in length and still more typically about 20 to 130 ribonucleotides in length, and adenine will predominate. Thus, poly(A) markers/ladders can contain, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more ribonucleotides.

Most of the ribonucleotides with be adenine. Following a string a adenine ribonucleotides, there will be a different ribonucleotide, such as guanine. For example, for a string of 10 to 25 adenines, there can be a guanine residue followed by another string of 10 to 25 adenines, etc. Strings of 19 adenine residues are exemplified herein. The poly(A) markers/ladders can contain other ribonucleotides, such as cytosine, uracil and N1-methyl-pseudouridine (U^{NIM}).

The mRNA constructs and poly(A) designs used herein are representative of current mRNA-based therapeutics. Further details of the disclosed methods and systems are provided below.

A. Method for Characterizing a Polyadenylation Region on mRNA Using Capillary Gel Electrophoresis (CGE)

In one aspect, a method for characterizing a polyadenylation region on mRNA using a CGE approach is provided. For example, a method for characterizing a polyadenylation region on mRNA may include adjusting mRNA concentration in an mRNA sample based upon mRNA length. The method for characterizing a polyadenylation region on mRNA may include treating the mRNA sample with RNase T1 to remove the coding region and allow the polyadenylated region to remain. The method for characterizing a polyadenylation region on mRNA may include incubating the RNase T1 treated sample with oligo-dT beads. The method for characterizing a polyadenylation region on mRNA may include eluting the polyadenylated region bound to the oligo-dT beads. The method for characterizing a polyadenylation region on mRNA may include removing residual salts from the elution of the polyadenylated region bound to the oligo-dT beads. The method for characterizing a polyadenylation region on mRNA may include analyzing, using capillary gel electrophoresis (CGE), the polyadenylated region after removing the residual salts from the elution of the polyadenylated region bound to the oligo-dT beads.

One or more of the following example features may be included. The step of removing residual salts from the elution may include using nuclease free water. The step of removing residual salts from the elution may include using a Zeba spin, dialysis, tangential flow flotation, or CAT cartridges. The step of analyzing the polyadenylated region may include using a binding buffer comprising, for example, 20 mM Tris-HCl, a pH of 7.5, 2 mM EDTA, and 1000 mM LiCl. The step of analyzing the polyadenylated region may also include using a separation buffer comprising Tris-Borate-Urea. The step of analyzing the polyadenylated region may further include using a wash buffer comprising, for example, 10 mM Tris-HCl, a pH of 7.5, 1 mM EDTA, and 150 mM LiCl. The method for characterizing a polyadenylation region on mRNA may further include, prior to analyzing the polyadenylated region, mixing the mRNA sample with mRNA ladders comprising poly(A) with guanine nucleotide spacers. The RNase T1 sample may comprise about 0.5M salt to allow binding to the oligo-dT beads. The step of removing residual salts from the elution may facilitate CGE. The step of analyzing the polyadenylated region may utilize an ultraviolet (UV) detector for detecting the polyadenylated region. The UV detector may use a light at a wavelength of 250 to 260 nm. The UV detector may use a light at a wavelength of 254 nm.

### B. Preparation of Messenger RNA

In the present inventions, mRNAs were synthesized using MegaScript T7 Transcription Kit (Thermo Fisher Scientific, Inc., MA, USA) with kit-supplied rATP, rCTP, rGTP, and N1-Methylpseudouridine-5′-Triphosphate (TriLink Biotechnologies, CA, USA) and CleanCap® Reagent AG (3′ OMe) Analog (TriLink Biotechnologies, CA, USA). The reaction mixtures were incubated at 37° C. for 2.5 hours and further incubated at 37° C. for 20 minutes in the presence of TURBO DNase I (Thermo Fisher Scientific, Inc., MA, USA). Synthesized mRNA products were purified with RNeasy Midi kit (QIAGEN, Inc., CA, USA) according to the manufacturer's protocol. The amounts of purified mRNAs were determined by Nano Drop (Thermo Fisher Scientific, Inc., MA, USA).

C. Preparation Steps to Purify Poly(A) from Full-Length mRNA (T1 Cleavage and Poly(A) Tail Isolation from mRNA)

For each of the different poly(A) designs (that is, designs A, B, and C), the IVT produced mRNA constructs, including EGFP, Fly Luciferase and Cas9 mRNA, were digested with an RNase T1 enzyme at a ratio of 33 pmole mRNA per 800 U T1 at 37° C. for one (1) hour.

During the digestion period, Dynabeads oligo-dT (25) (200 μL beads/2 μg total mRNA) were conditioned by washing two times (2×) with 1× binding buffer (10 mM Tris-HCl, pH 7.5, 1 mM EDTA, 500 mM LiCl).

Upon completion of the T1 digestion period, the digested mRNA was mixed at a 1:1 (v/v) ratio with 2× binding buffer (20 mM Tris-HCl, pH 7.5, 2 mM EDTA, 1000 mM LiCl) and then added to Dynabeads Oligo-dT (25) conditioned with 1× binding buffer (10 mM Tris-HCl, pH 7.5, 1 mM EDTA, 500 mM LiCl) and incubated at RT for one (1) hour to allow the isolated poly(A) tail to bind to the beads.

After the incubation, poly(A)-bound beads were washed 2× with washing buffer (10 mM Tris-HCl, pH 7.5, 1 mM EDTA, 150 mM LiCl). Poly(A) was then released from the beads by adding nuclease free (NF) water to the beads, heated to 80° C. for two (2) minutes, and then incubated again at RT for 30 minutes.

During the 30 minute incubation period, Zeba filter columns were equilibrated by adding 300 μL of NF water to the filters and then spun for one (1) minute at 1450 g speed. The equilibration was repeated three times (3×).

After incubation, the supernatant from the beads was removed and then added to the Zeba filters (7K MWCO) and spun down for two (2) minutes at 1450 g speed to remove any residual salt.

Notably, the samples can be injected via reverse polarity electrokinetic injection, where a voltage is applied to the sample for a period of time, essentially selectively injecting the negatively charged poly(A) into the capillary. With the residual salt from the DynaBead purification (LiCl), the negative counter ion from the salt will also be injected, reducing the amount injected of the poly(A). This will result in a significantly decreased intensity and, ultimately, sensitivity of this method to measure the full distribution of the poly(A) length. Accordingly, the removal of the residual salt is essential to the efficacy of the method of the present inventions. Other desalting methods include dialysis, tangential flow flotation, CAT cartridges or Sep-Pak C18.

The maximum concentration of poly(A) after purification is complete is 24 ng/uL (with 100% recovery). Unless otherwise mentioned, all incubation and digestion steps in the present inventions for characterizing a polyadenylation region on mRNA were done at room temperature (RT) and on a hulu (shaker/rocker/mixer).

### D. General Workflow of the Poly(A) Isolation and Downstream Characterization

The workflow for poly(A) tail isolation is illustrated in FIG. 1B (SEQ ID NOS: 4 to 11). As illustrated, a concentration of mRNA (sequence and length independent) can be normalized against the poly(A) percent based on the different length of mRNA constructs. The mRNA is thereafter subjected to RNAase T1 enzyme (Thermo Fisher) digestion followed by oligo-dT bead isolation of poly(A) tail. For example, for each poly(A) sample, 33 pmol of mRNA was subjected to 800 U of RNAase T1 treatment for one (1) hour at 37° C.; wherein “U” is a generic unit to measure enzymatic activity and issued at 1000 U per μl as provided in the tubes, and 33 pmol is the absolute amount in moles for mRNA used (that is, 100 μl of 0.33 uM of mRNA). Both T1 digestion condition and oligo-dT isolation of poly(A) tail were optimized to achieve greater than 50% recovery of the poly(A) tail. Based on previous studies, there is no bias of oligo-dT beads for poly(A) longer than 20A. As such, the recovered poly(A) tail was expected to be representative of the poly(A) tail from the mRNA construct.

The isolated poly(A) tail was then ready for downstream characterization. The CGE method was compared with two LC based methods: AEX-LC and IP-RP-LC to compare: 1) resolution and sensitivity of method; and 2) case of implementing as release method.

### E. CGE Characterization of Different Poly(A) Designs

Capillary gel electrophoresis (CGE) has been used extensively for the quantitative analysis of oligonucleotide therapeutics in both preclinical and clinical studies since the 1990s. The success of CGE is based on its extraordinary resolving power compared to traditional liquid chromatography methods (n/n+1 resolution). The single base resolving power and the case for utilizing CGE in release testing makes it an attractive approach for poly(A) tail length characterization for monitoring CQA of mRNA-based therapeutics. The SCIEX PA800 plus system, given its flexibility in customizing different components (capillary length, matrix gel composition, injection voltage, etc.), was chosen to be used in this CGE method development.

### III. Methods of Distinguishing Between Intact and Degraded mRNA

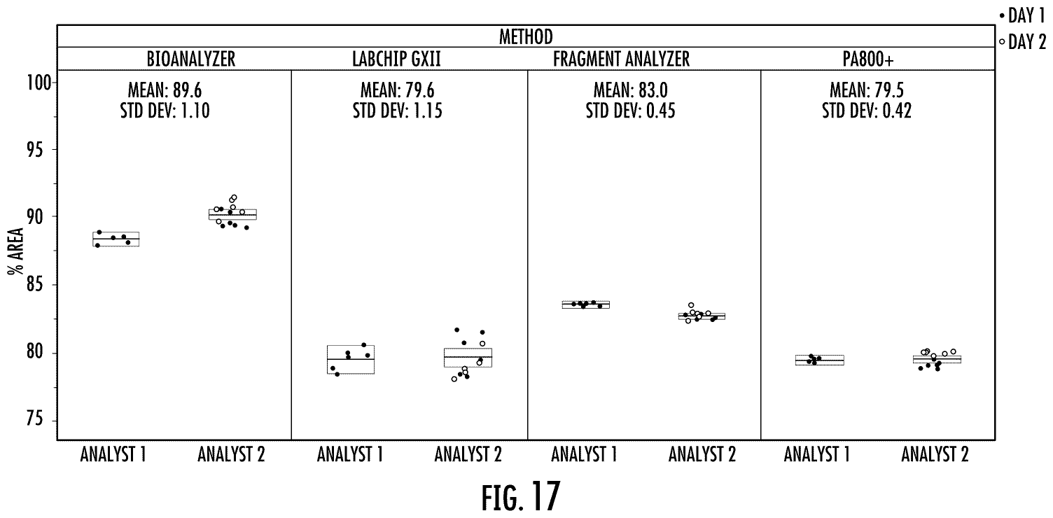

An intact mRNA molecule is schematically depicted in FIG. 18. An intact mRNA would be considered full length, and comprises a 5′ Cap region, a 5′ untranslated region (UTR), an open reading frame, a 3′ UTR and a poly(A) tail). Intact mRNAs degrade in nature, and typically first by shortening of the poly(A) tail and then removal of the 5′ Cap region. Thereafter, the mRNA tends to lose nucleotides in the 5′ to 3′ direction.

Messenger RNAs in in vitro setting can degrade based upon environmental conditions, such as subjection to multiple freeze/thaw cycles or storage at elevated temperatures, such as 25° C., 30° C., 35° C., 40° C., 45° C., 50° C., 55° C., 60° C., 65° C., 70° C., 75° C., 80° C., 85° C., 90° C., 95° C., 100° C. or more. Such degradation of the mRNA molecules can occur by shortening of the poly(A) tail, removal of the 5′ Cap region, and loss in the coding region, as discussed above, and in a more stochastic manner of sequence fragmentation.

The present inventions provide for the use of CE, MCE and CGE for detection of intact and degrade mRNA in samples. Example 8 sets forth data that validates CE and MCE for use in mRNA validation. Examples 1-7 present data showing the effectiveness of CGE. Example 8 shows that CE-based method achieved higher resolution than IPRP-LC methods. Example 9 sets for methods for evaluating various mRNA preparations using representative CE and MCE systems.

The inventions are further described by the following Examples, which do not limit the inventions in any manner. The order of performance of the below examples can be altered or combined as determined by the person of skill in the art in view of the teachings and data contained herein.

### EXAMPLES

### Example 1: CGE-UV Profile of the Three Different mRNA Poly(A) Designs of Different mRNA Constructs

To obtain optimal resolution for the three different mRNA poly(A) designs, the following factors were optimized: injection mode, injection amount, voltage for separation and capillary length. Electrokinetic injection was compared with pressure injection and among the two, kinetic injection provided much better resolution.

All 9 poly(A) samples, containing the three different poly(A) designs and from three different mRNA constructs, were analyzed using the CGE method of the present invention. For each poly(A) sample to be analyzed by the CGE method, 33 pmol of mRNA was subjected to 800 U of RNAase T1 treatment for one (1) hour at 37° C.

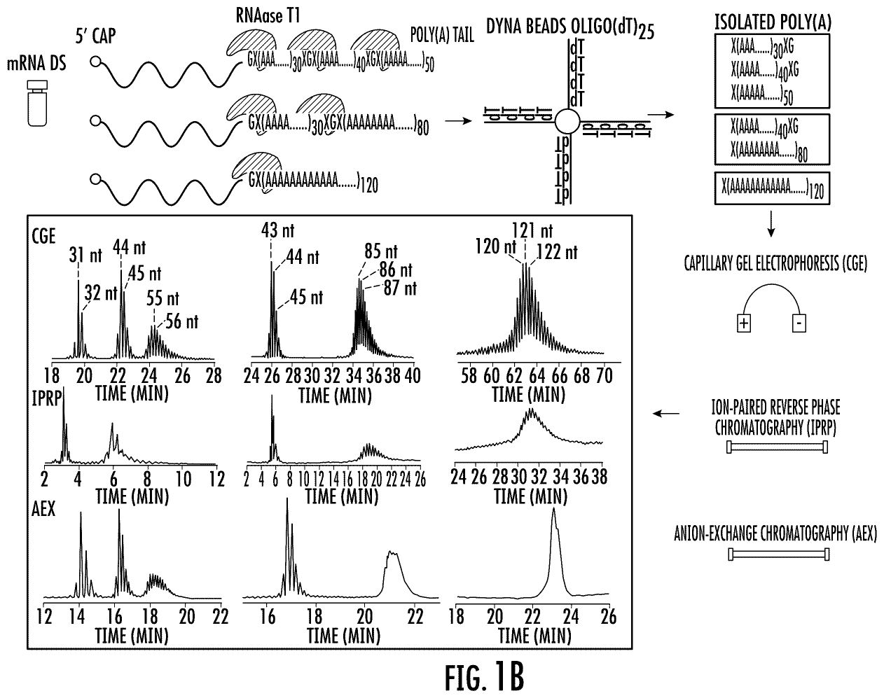

The poly(A) peak distribution can be found in the electropherogram results shown in FIGS. 2A-2F, 3A-3F, and 4A-4F, wherein each respective figure depicts the peak profile for each mRNA construct and for each of the three poly(A) designs. Regardless of poly(A) design, it was found that the poly(A) length was always a distribution rather than a given length, which was consistent with previous reports using LC-MS or NGS methods.

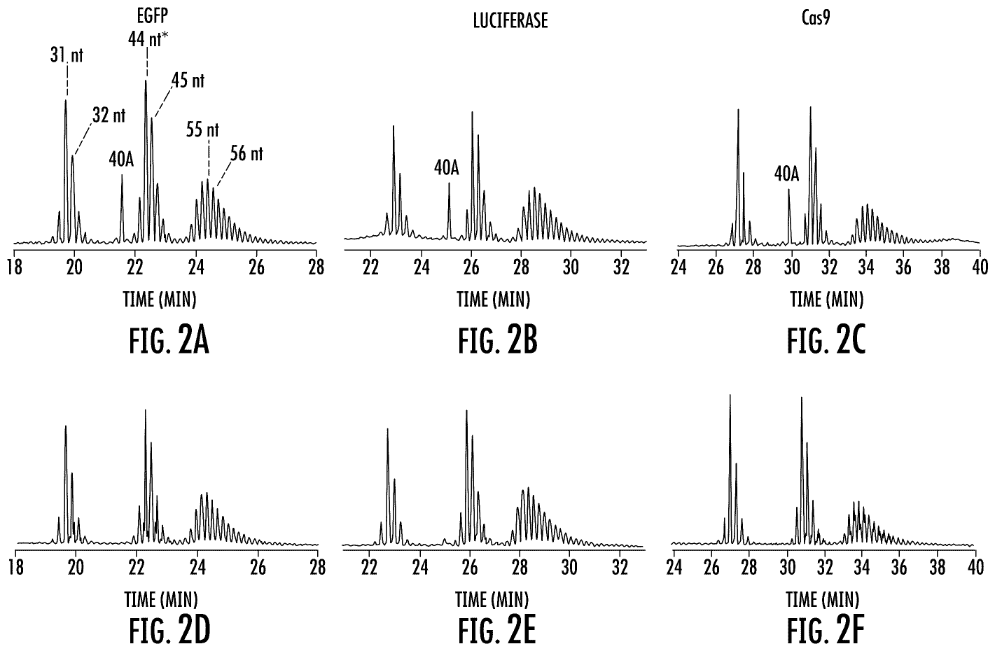

For poly(A) design A, three groups of distributions were observed (See, FIGS. 2A-2F) and was consistent with the three segments of poly(A) in the design. For poly(A) design B, two groups of distributions were observed (See, FIGS. 3A-3F), which was also consistent with the two segments of poly(A) in the design. For all three poly(A) designs (that is, A, B, and C in FIG. 1A), an n/n+1 resolution was achieved using highly similar CGE running condition, thus providing invaluable information for the poly(A) length heterogeneity.

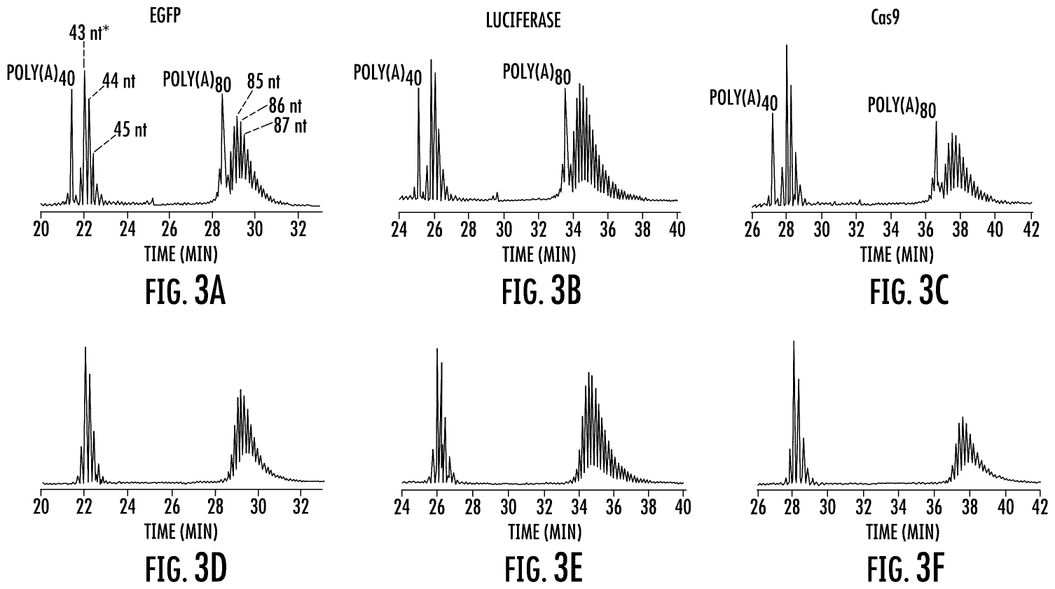

For poly(A) design A, six different lengths were separated for 30-A segment. For the 40-A, seven to eight different lengths were separated and for the 50-A, at least 15 different lengths could be identified from the chromatogram. For poly(A) design B, at least 7 different lengths were observed for the 40-A segment and more than 20 were observed for the 80-A segment. For poly(A) design C, more than 30 different lengths were observed (See, FIGS. 4A-4F).

Because the poly(A) s for the three different mRNA constructs were added using the same set of primers, the poly(A) length distribution profiles were highly similar among three mRNA constructs. To achieve relative similar intensity of the peaks, mRNA digestion amounts may require adjusting based on the percentage of poly(A) length compared with the full length of mRNA. For example, a 120-A poly(A) account for ˜ 12% of EGFP mRNA construct while the same poly(A) tail is only ˜3% of Cas9 mRNA.

### Example 2: AEX Characterization of Different Poly(A) Designs

Isolated poly(A) from mRNA after T1 cleavage was analyzed by AEX using a Thermo Fisher DNApac PA200 RS (4.6×150 mm) column on an Agilent 1290 Infinitely 2DLC system (Agilent Technologies) utilizing the ID only. Mobile phase A (MPA) contained 40 mM Tris in nuclease free water with pH adjusted to 8.0. Mobile phase B (MPB) contained 40 mM Tris and 1 M NaCl in nuclease free H2O with pH 8.0. Both mobile phases were prepared using nuclease free Tris stock at pH 8.0. The flow rate for AEX is 0.5 ml/min, and the gradient consisted of 41-80% MPB from 0 to 27.3 min, maintained at 80% MPB until 29 min. MPB was reduced to 41% from 29 to 30 min and then maintained at 41% until the end of the 50-minute gradient. The column was equilibrated for 20 minutes at 41% MPB before injecting each sample. The column temperate was set at 30° C. Data were recorded using a DAD detector at wavelength of 260 nm, 280 nm, and 215 nm.

Separation tasks become more challenging with increasing number of nucleotides for charge-based separation. For example, 10 nt versus 11 nt differs by 10% in total negative charge, while 100 nt versus 101 nt differs by only 1% in total negative charge. However, AEX has been shown to provide n/n+1 resolution for oligonucleotide up to 60-nt long.

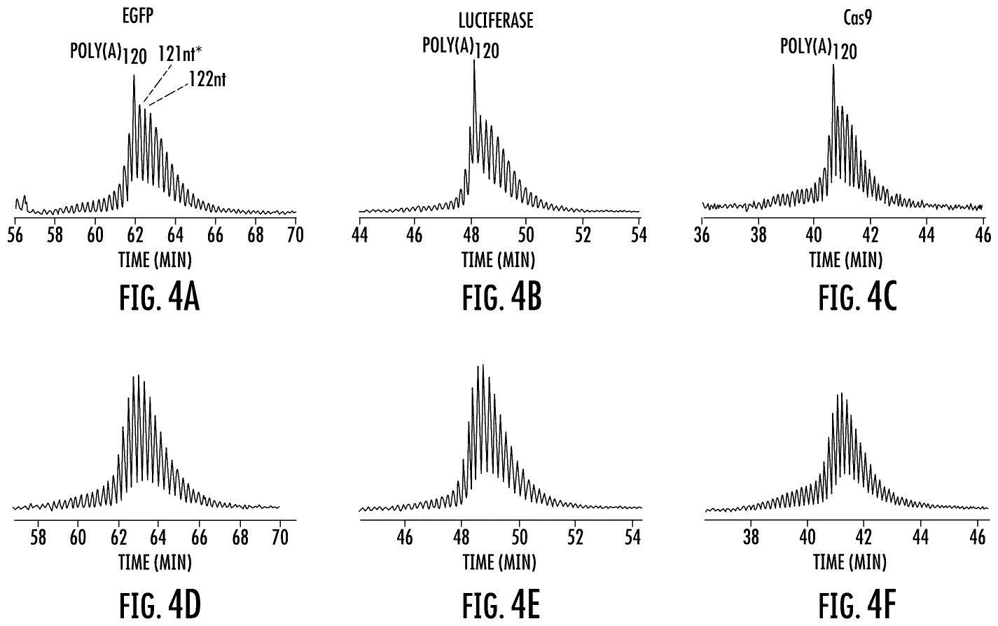

A standard AEX separation used for oligonucleotide analysis was applied to characterize the poly(A) length and size heterogeneity from the three different poly(A) designs. (See, FIGS. 5A-5C). Because all three mRNA constructs showed highly similar poly(A) length and distribution, Cas9 mRNA was used an example to test the resolution of the AEX-LC method.

For poly(A) design A, three groups of peaks were observed, corresponding to the 30-A, 40-A and 50-A segments separated by the G-containing spacer in the poly(A) design. N/n+1 resolution was achieved for all three groups given the length is in the optimal range. For poly(A) design B, two groups of peaks were observed, consistent with the 40-A and 80-A separated by the G containing spacer in the design. For the 40-A group an n/n+1 resolution was achieved; however, only partially achieved for the 80-A group even after optimizing the injection amount among other conditions. For poly(A) design C, in which there was an expected length of 120-A, only a single peak was observed without any n/n+1 resolution; although, this result was expected given the charge differences were smaller than 1% for this length of poly(A).

Prior methods used to measure poly(A) tail (for example, LC-MS, next-generation sequencing, etc.), have demonstrated that mRNA poly(A) tail always contains a distribution of lengths rather than a single tail length, regardless of whether the mRNA was from a cell or IVT-reaction.

The AEX method exemplified herein, which is provided only for comparative purposes to the improved CGE methodology of the present inventions, proved to be consistent with prior methods, even poly(A) design C, which although lacking the n/n+1 resolution, the broad peak observed in poly(A) design C indicated a distribution of length rather than a single, defined length of poly(A) tail. Notably, the relative peak intensity profiles between the AEX-LC and the CGE methods are highly similar for poly(A) design A and B.

### Example 3: Poly(A) Ladder Spike in the Three Different mRNA Poly(A) Designs

Given the baseline n/n+1 resolution demonstrated in the CGE methods for all mRNA constructs with different poly(A) designs, it may be possible to directly read out the length of poly(A) on the electropherogram if appropriate poly(A) ladders can be incorporated.

A series of poly(A) ladders have been designed, synthesized, and used to enable direct read-out of poly(A) length. (See, Table 2 and SEQ ID NOS: 17 to 22).

TABLE 2
Synthetic poly(A) ladder sequence

| Marker* | Length (nts) | Sequence |
|---|---|---|
| Poly(A)₂₀ | 20 | rArArArArArArArArArArArArArArArAr |
| | | ArArArG |
| Poly(A)₄₀ | 40 | rArArArArArArArArArArArArArArArAr |
| | | ArArArGrArArArArArArArArArArArAr |
| | | ArArArArArArArG |
| Poly(A)₆₀ | 60 | rArArArArArArArArArArArArArArArAr |
| | | ArArArGrArArArArArArArArArArArAr |
| | | ArArArArArArArGrArArArArArArArAr |
| | | ArArArArArArArArArArArG |
| Poly(A)₈₀ | 80 | rArArArArArArArArArArArArArArArAr |
| | | ArArArGrArArArArArArArArArArArAr |
| | | ArArArArArArArGrArArArArArArArAr |
| | | ArArArArArArArArArArArGrArArArAr |
| | | ArArArArArArArArArArArArArArArG |
| Poly(A)₁₀₀ | 100 | rArArArArArArArArArArArArArArArAr |
| | | ArArArGrArArArArArArArArArArArAr |
| | | ArArArArArArArGrArArArArArArArAr |
| | | ArArArArArArArArArArArGrArArArAr |
| | | ArArArArArArArArArArArArArArArGr |
| | | ArArArArArArArArArArArArArArArAr |
| | | ArArArG |
| Poly(A)₁₂₀ | 120 | rArArArArArArArArArArArArArArArAr |
| | | ArArArGrArArArArArArArArArArArAr |
| | | ArArArArArArArGrArArArArArArArAr |
| | | ArArArArArArArArArArArGrArArArAr |
| | | ArArArArArArArArArArArArArArArGr |
| | | ArArArArArArArArArArArArArArArAr |
| | | ArArArGrArArArArArArArArArArArAr |
| | | ArArArArArArArG |

The ladder mixture may be ran after each poly(A) run or may spike-in into the isolated poly(A) and co-migrate. In the first approach, six (6) poly(A) ladders were mixed in a certain ratio given their relative UV260 absorption. For the second approach, depending on the length of poly(A) for analysis, a certain ladder or set of ladders were spiked into the isolated poly(A) and compared via electropherogram to a sample without spiking. For each gel fill, up to five (5) injections of samples were completed without significant shift in the migration time. A typical set of injections in this case included: three replicates of poly(A) isolated from sample of interest, one injection of six (6) poly(A) ladder mixture and one ladder-spiked poly(A) tail isolated from sample.

For the second approach to work, the ratio of dilution for the spiked ladder needed to give enough signal of the spiked ladder without compromising the resolution or masking the actual poly(A) sample signal. The appropriate ratio of dilution are displayed in Table 3.

TABLE 3
Dilution ratio of poly(A) ladders for spiking in poly(A) samples

| | Stock concentration | Dilution factor when |
|---|---|---|
| Poly(A) ladder | (mg/ml) | used in spike* |
| 20-A | 1.0 | Not used in spike in this study |
| 40-A | 1.0 | 1200-fold |
| 60-A | 1.0 | 900-fold |
| 80-A | 1.0 | 200-fold |
| 100-A | 1.0 | Not used in spike in this study |
| 120-A | 1.0 | 190-fold or 240-fold |

*single or combined ladder(s) spiking utilizing the same dilution factor.

Notably, the ratio should be adjusted based on the yield of poly(A) ladder from the solid-state synthesis.

The two approaches were compared under poly(A) design A. The isolated poly(A) from three different mRNAs were analyzed using the set of injections mentioned above (triplicates+6-ladder mixture+spike); the alignment of the poly(A) marker being found in Table 2. In this case, 40-A ladder spiked into the samples given the length of the three poly(A) segments in design A.

The 40-A ladder spiked in the sample aligned perfectly with the 40-A in the 6-ladder mixture, suggesting there was no interference of the migration of the ladder once spiked into poly(A) sample. (See, Table 2). Given the n/n+1 resolution in the CGE method, the most abundant peak in each poly(A) segment were read from the electropherogram by counting from the spiked ladder. (See, FIG. 2A to FIG. 4F). Two ladders (40-A and 80-A) were spiked into the poly(A) design B while one ladder (120-A) was spiked into poly(A) design C from all three different mRNA constructs. An additional ladder (60-A) spiked into the sample containing poly(A) design A, serving as further confirmation of the poly(A) length (See, Table 2). Alignment between the 60-A ladder spiked in the sample and the 60-A in the 6-ladder mixture further confirmed that the migration time of the ladders were the same regardless of whether it was spiked into the actual sample or was part of the 6-ladder mixture.

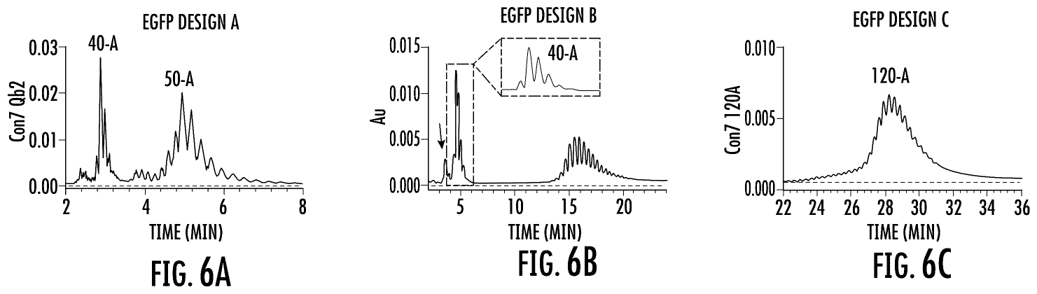

Similar comparisons were performed for other ladders (80-A and 120-A), finding no difference in migration time observed. (See, FIGS. 7A-7C, poly(A) design B and C). Provided the convenience and ease of the second approach, the ladder spike-in was adopted for detailed analysis of poly(A) design B and C, and recommended for use in further studies. Two ladders (40-A and 80-A) were spiked into the poly(A) design B while one ladder (120-A) was spiked into poly(A) design C from all three different mRNA constructs. The most abundant peak each segment and designs were summarized in Table 4.

TABLE 4
Expected length of poly(A) and segments and experimental
determined length (based on the highest peak)

| mRNA | Poly(A) | Expected Poly(A) | CGE determined | CGE determined most |
|---|---|---|---|---|
| construct | design | length* | length range | abundant peak |
| EGFP | A | 35, 46, 53 | 29-35, 41-48, 51-66 | 31, 44, 55 |
| EGFP | B | 47, 83 | 41-48, 80-102 | 43, 85 |
| EGFP | C | 122 | 106-135 | 121 |
| Luciferase | A | 35, 46, 53 | 29-35, 41-48, 51-66 | 31, 44, 55 |
| Luciferase | B | 47, 83 | 41-48, 80-102 | 43, 85 |
| Luciferase | C | 122 | 106-135 | 121 |
| Cas9 | A | 35, 46, 53 | 29-35, 41-48, 51-66 | 31, 44, 55 |
| Cas9 | B | 47, 83 | 41-48, 80-102 | 43, 85 |
| Cas9 | C | 122 | 106-135 | 121 |

*Expected poly(A) length is based on the primary sequence of linearized template DNA.

### Example 4: IP-RP Characterization of Different Poly(A) Designs

Ion-paring reverse phase (IP-RP) liquid chromatography is routinely used for characterization and separation of oligonucleotide, as well as purity analysis of full length IVT-produced mRNA.

To achieve longer oligonucleotide analysis, including poly(A), an IP-RP method, which is described herein only for comparative purposes to the improved CGE methodology of the present inventions, may implement hydrophobic alkylamines (hexylamine, octylamine, high concentration of IP agent in the mobile phase (for example, 50-100 mM), and columns (that is, long columns packed with sub two-micron particles).

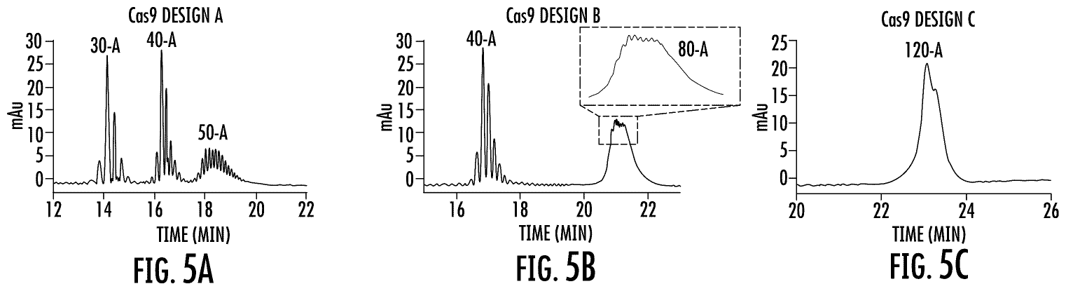

To assess the resolution of the IP-RP method, a poly(A) ladder mixture (containing 20A, 40A, 60A, 80A, 100A and 120A) was analyzed using the same protocol in Example 1. It was clear that the IP-RP-LC (OAA method) and gradient were optimized for longer poly(A), with particular dampening in the signal for longer poly(A) ladders, including 80A, 100A and 120A. N/n+1 resolution was achieved using the IP-RP-LC (OAA method) for the 80A segment of poly(A) design B and partially for 120A. (See, FIGS. 6A-6C).

### Example 5: Isolated Poly(A) Under Poly(A) Design a from Three Different mRNA Constructs with Poly(A) 40 and Poly(A) 60 Combined Spike-In

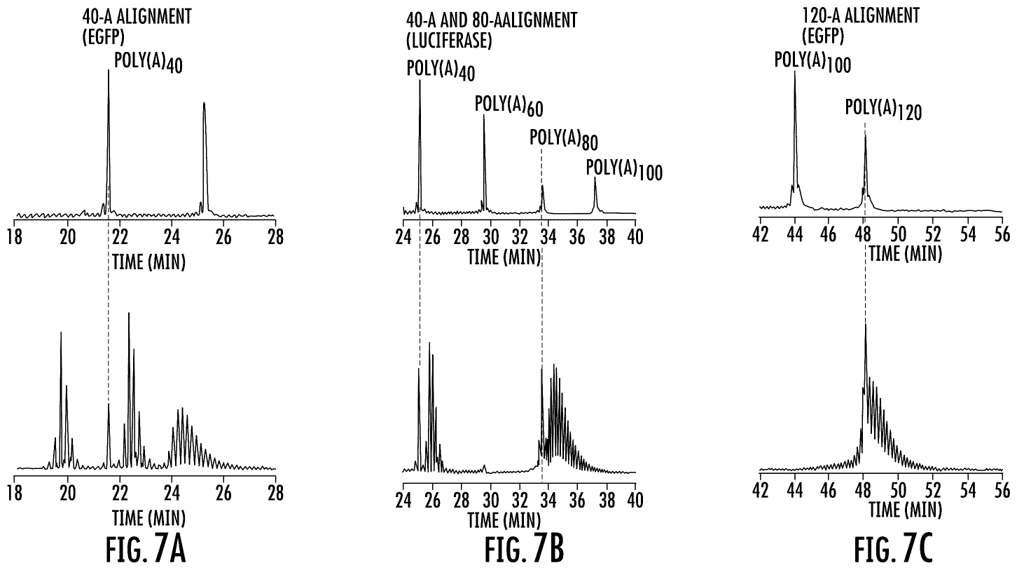

Isolated poly(A) was analyzed according to the CGE method of the present inventions under poly(A) design A for three different mRNA constructs with poly(A) 40 and poly(A) 60 combined spike-in. The isolated poly(A) peak distribution can be found in the electropherogram results shown in FIGS. 8A-8C, wherein each respective figure depicts the peak profiles of EGFP mRNA, Fly Luciferase mRNA, and Cas9 mRNA, respectively, with poly(A) 40 and poly(A) 60 combined spike-in. The results indicated that the poly(A) segments showed highly similar length distribution among the three mRNA constructs.

### Example 6: Isolated Poly(A) Under Three Different Poly(A) Designs Using CGE Triplicates of Injections for Three Different mRNA Constructs

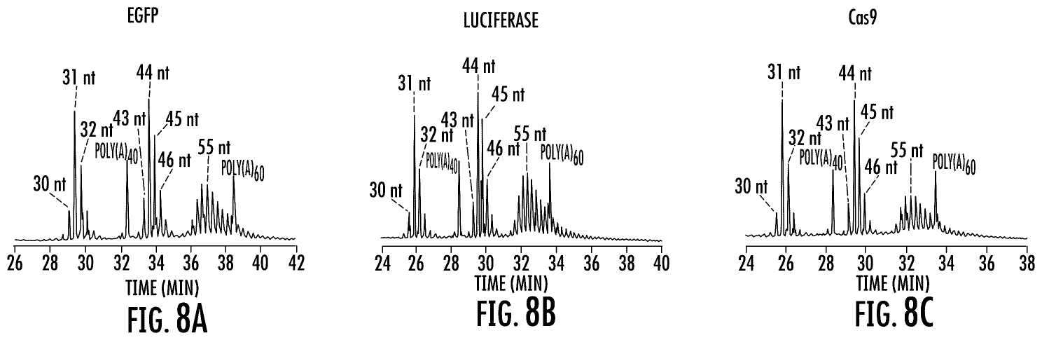

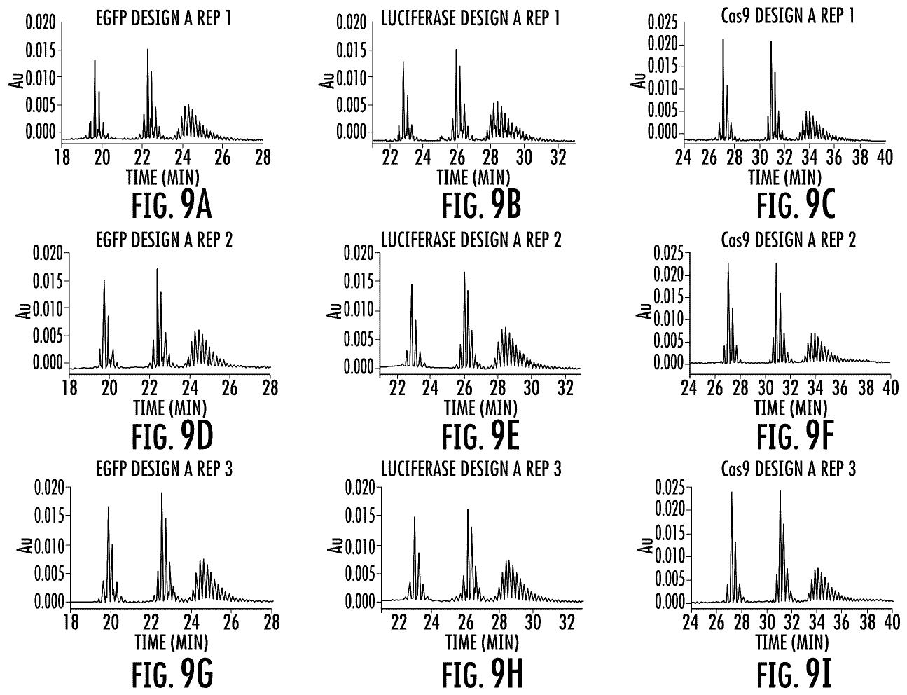

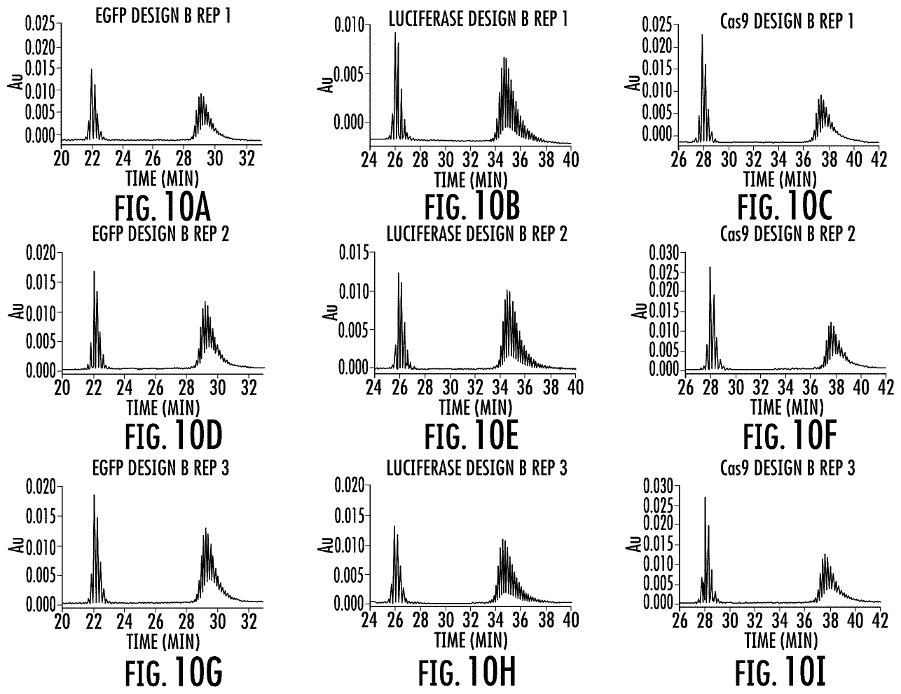

Nine poly(A) samples were analyzed according to the CGE method of the present inventions under poly(A) designs A, B, and C for three different mRNA constructs. The isolated poly(A) peak distribution under poly(A) design A can be found in the electropherogram results shown in FIGS. 9A-9I, wherein each respective row are the measurements collected from an iteration of the CGE triplicate for the peak profiles of EGFP mRNA, Fly Luciferase mRNA, and Cas9 mRNA, respectively. The isolated poly(A) peak distribution under poly(A) design B can be found in the electropherogram results shown in FIGS. 10A-10I, wherein each respective row are the measurements collected from an iteration of the CGE triplicate for the peak profiles of EGFP mRNA, Fly Luciferase mRNA, and Cas9 mRNA, respectively. The isolated poly(A) peak distribution under poly(A) design C can be found in the electropherogram results shown in FIGS. 11A-11I, wherein each respective row are the measurements collected from an iteration of the CGE triplicate for the peak profiles of EGFP mRNA, Fly Luciferase mRNA, and Cas9 mRNA, respectively.

### Example 7: Poly(A) Marker Migration Profiles

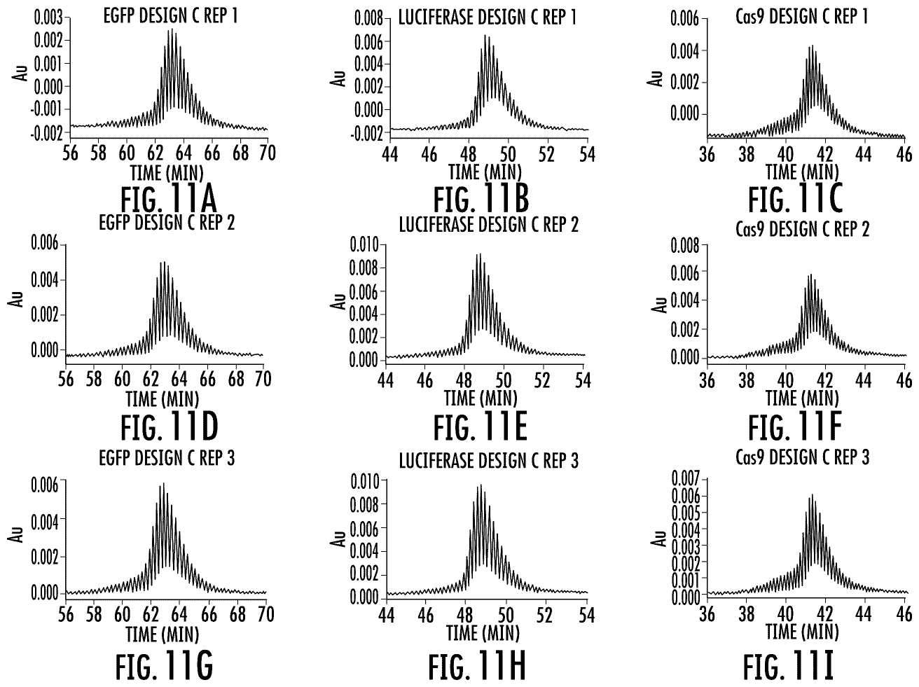

Six (6) poly(A) marker migration profiles were analyzed according to CGE-UV methodology of the present inventions. The results of the analysis can be found in the electropherogram shown in FIG. 12, which indicates that a wide separation of markers was observed for all six lengths when using CGE.

### Example 8: CE-Based Methods Show Higher Resolution than IPRP-LC Methods when Analyzing mRNA

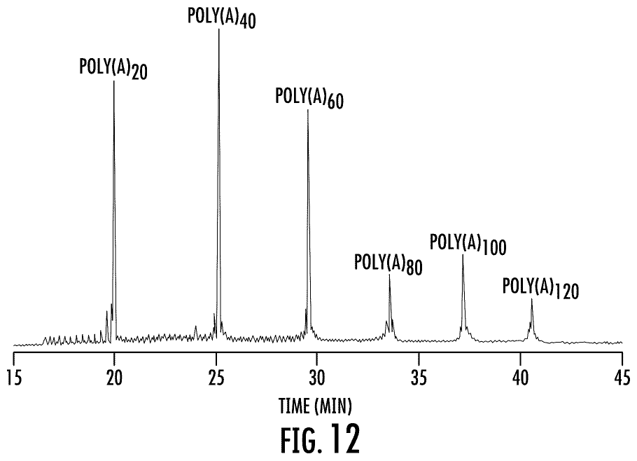

FIGS. 13A-13B compare IPRP-LC and CE methods in terms of resolution using EGFP mRNA. FIG. 13A utilized IPRP-LC and shows a relatively broad peak and a small right-side shoulder. FIG. 13B shows a CE method yields a much narrower peak.

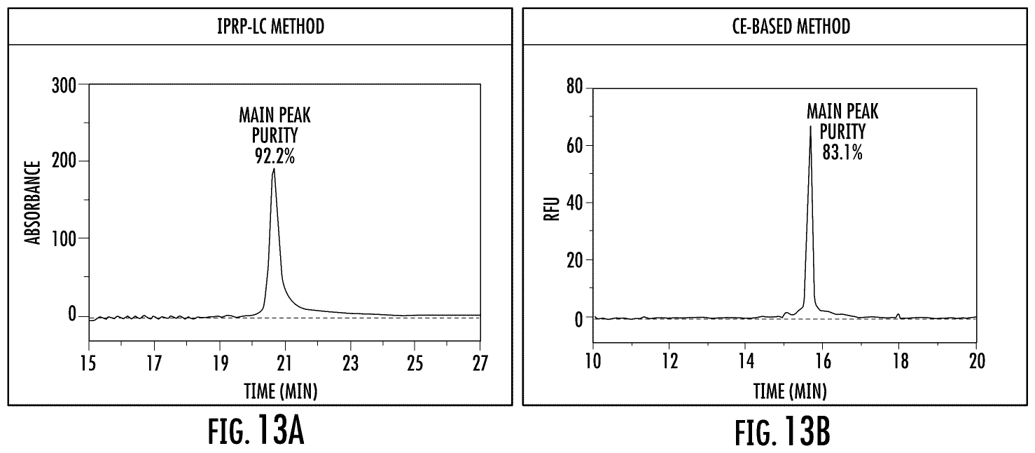

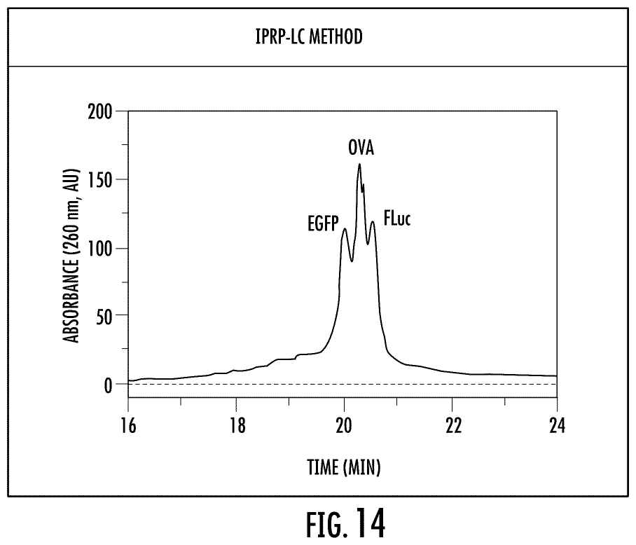

FIG. 14 depicts absorbance data from a IPRP-LC, and shows limited resolution with EGFP, OVA, and FLuc. FIGS. 15A-D show data using capillary electrophoresis with different types of analyzers. FIG. 15A uses a Bioanalyzer, which is an MCE device. FIG. 15B uses a LabChip GXII, which is an MCE device. FIG. 15C uses at Fragment Analyzer, which is a CE device. FIG. 15D uses at PA800 Plus, which is a CE device. FIG. 15A-D show distinctively better resolution between EGFP, OVA and FLuc than FIG. 14 obtained using IPRP-LC. PA800 Plus showed the best resolution. See FIG. 15D.

The electrophoresis can be conducted using standard buffers and conditions.

Analysis of mRNA samples may utilize a fluorescence detector for detecting the mRNA integrity. The fluorescence detector may use light at a wavelength of 470 to 500 nm. Emission wavelengths of 480 to 540 nm can be used.

The sizes of each mRNA are shown in Table 5.

TABLE 5

| mRNA | Approximate Length (nt) |
|---|---|
| EGFP | 1000 |
| OVA | 1500 |
| FLuc | 2000 |
| Cas9 | 4500 |

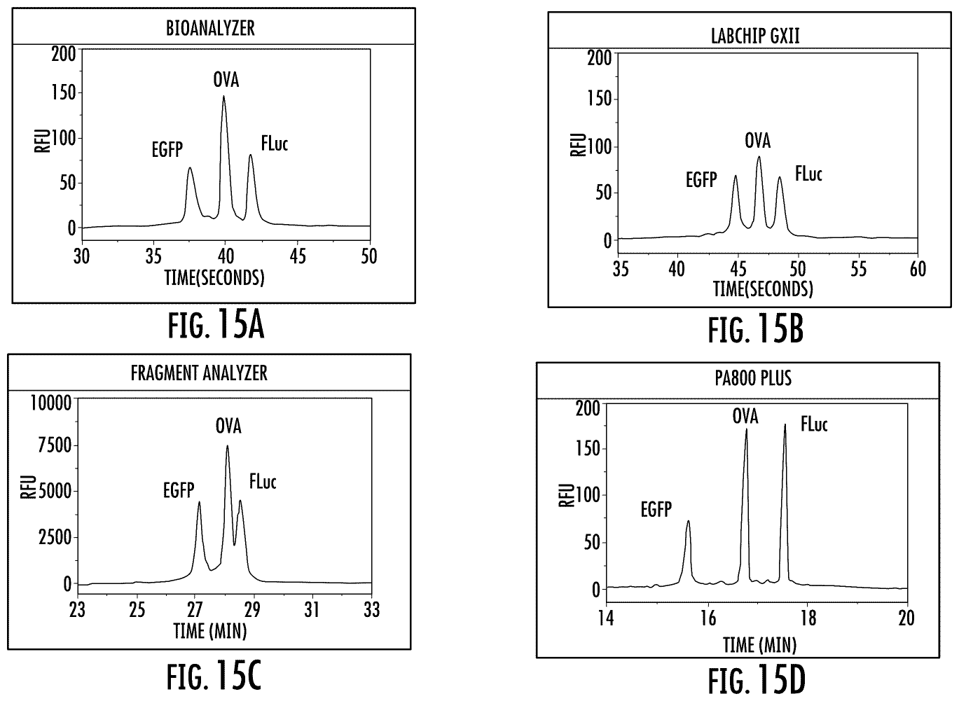

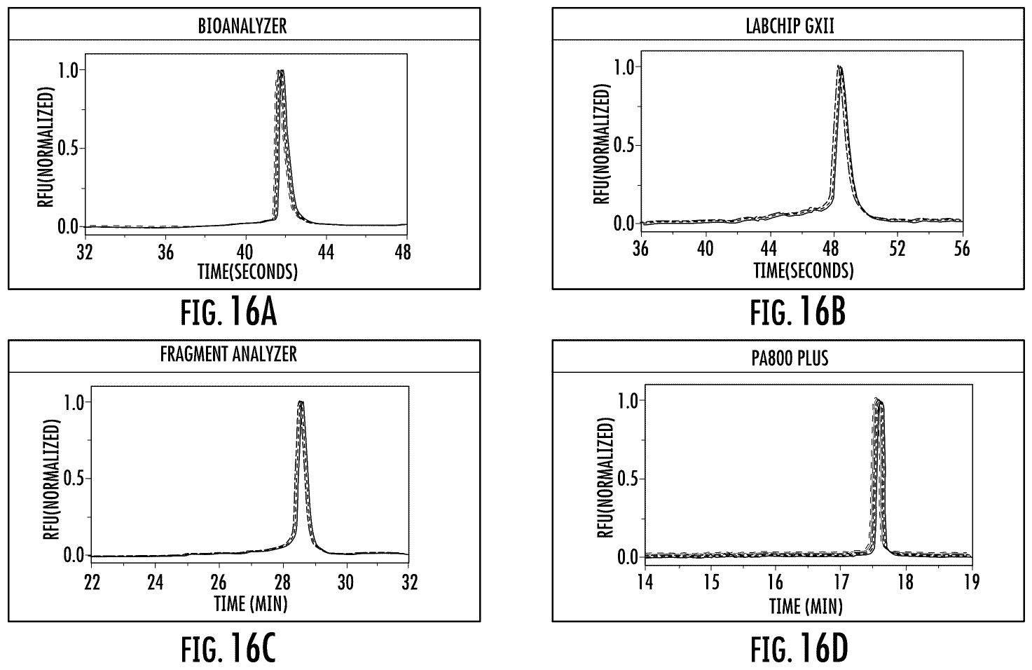

Turning to FIGS. 16A-16D, which depict RFU vs time data for 6 replicate injections of FLuc. This data shows the repeatability of the analyses using the Bioanalyzer (MCE), LabChip GXII (MCE), Fragment Analyzer (CE) and PA800 Plus (CE). FIG. 17 depicts percent (%) area at day 1 and day 2 using 6 replicate injections using FLuc mRNA, two different analysts and four types of analyzers, namely Bioanalyzer (MCE), LabChip GXII (MCE), Fragment Analyzer (CE) and PA800 Plus (CE).

The data show that CE and MCE performed with a variety of analyzers using differently sized RNA shows a high degree of repeatability across multiple injections, analysts and days.

### Example 9: CE and MCE can Distinguish Between Intact and Degraded mRNA

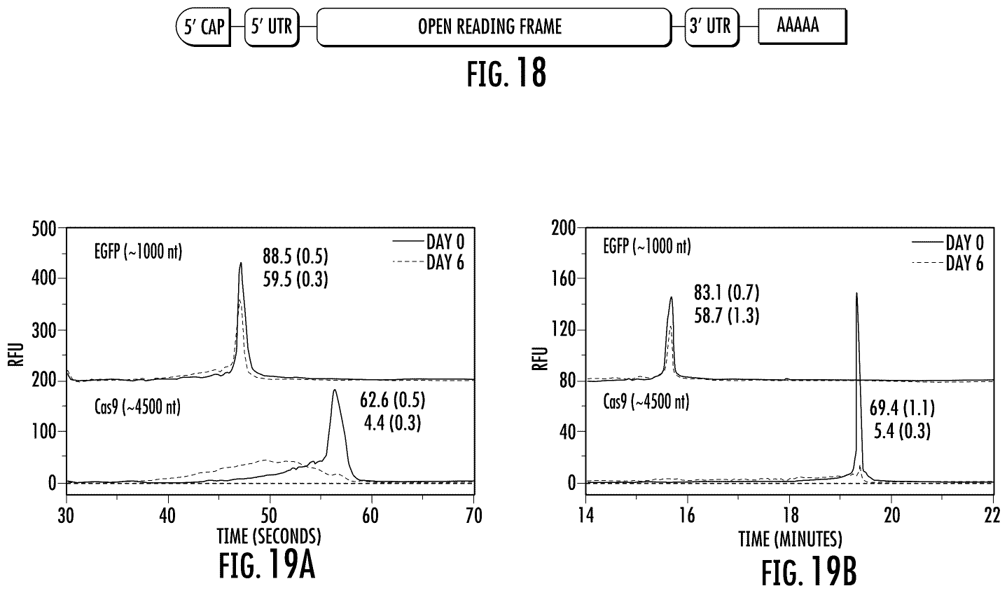

In this example, relatively small and relatively large mRNA were subject to condition that degrade mRNA, namely incubation at 40° C. for 6 days. FIGS. 19A-19B depict RFU vs time data using EGFP (top) and Cas9 (CRISPR Associated Protein 9 mRNA) (bottom) at Day 0 (blue) and Day 6 at 40° C. (red) to distinguish between intact mRNA and degraded mRNA. EGFP contains about 1000 nucleotides, and Cas9 contains about 4,500 nucleotides.

FIG. 19A shows RFU vs. time (seconds) separated using LabChip GXII (MCE). At Day 0, both EGFP and Cas9 show clearly delineated peaks. At Day 6 at 40° C., EGFP shows smaller perk indicating that 59.5% of EGFP transcripts remains intact. However, Day 6 at 40° C. shows that only 4.4% of Cas9 transcripts remain intact, and thus the vast majority is degraded.

FIG. 19B is data from the PA800 Plus (CE), and depicts the same phenomenon. At Day 6 at 40° C., EGFP had 58.7% of transcripts intact, whereas the Cas9 only had 5.4% of transcripts intact. Thus, the larger Cas9 is more susceptible to degradation than the smaller EGFP.

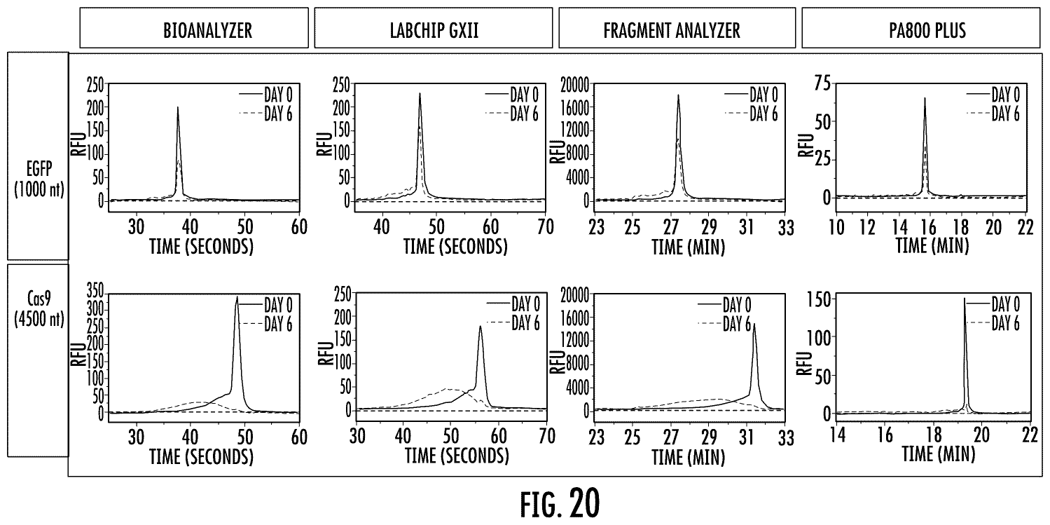

FIG. 20 depicts RFU vs. time data using EGFP (top) and Cas9 (bottom) using different types of analyzers, namely Bioanalyzer (MCE), LabChip GXII (MCE), Fragment Analyzer (CE) and PA800 Plus (CE). The data are consistent across orthogonal CE and MCE methods.

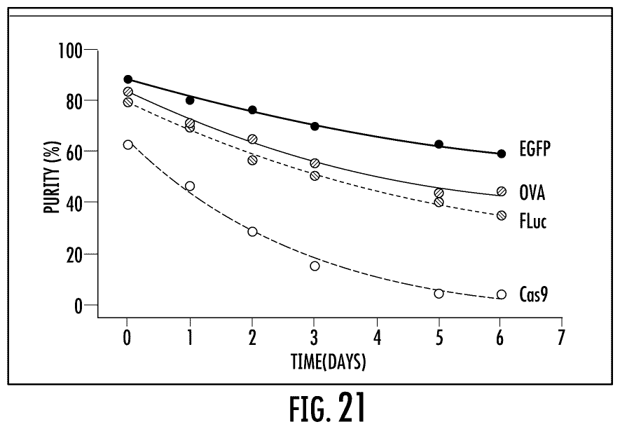

FIG. 21 depicts purity (%) vs. time (days) for EGFP, OVA, FLuc and Cas9 showing mRNA degradation over time. A LabChip GXII (MCE) was used for separation. The forced degradation results were sufficiently quantitative to accurately fit to a decay function for calculating half-life of mRNA. See Table 6 below.

TABLE 6

| | Approximate | Half-life |
|---|---|---|
| mRNA | Length (nt) | (days) |
| EGFP | 1000 | 10.5 |
| OVA | 1500 | 6 |
| FLuc | 2000 | 5 |
| Cas9 | 4500 | 1.6 |

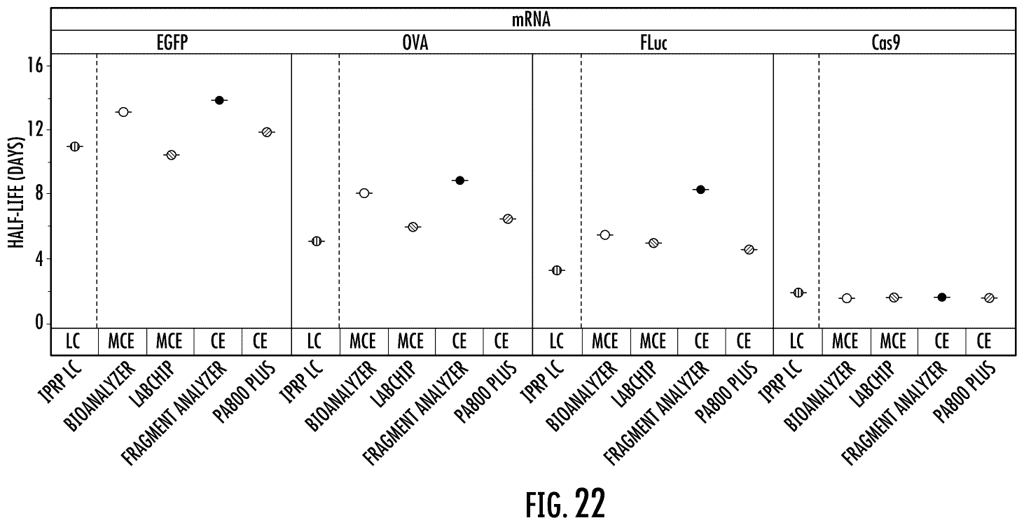

FIG. 22 depicts half-life data for EGFP, OVA, FLuc and Cas9 using five different analyzers, namely an IPRP, Bioanalyzer (MCE), LabChip GXII (MCE), Fragment Analyzer (CE) and PA800 Plus (CE). The forced degradation results were sufficiently quantitative to accurately fit to a decay function for calculating half-life of mRNA. The larger the mRNA, the shorter the half-life.

### Example 10: Optimization of Size Markers

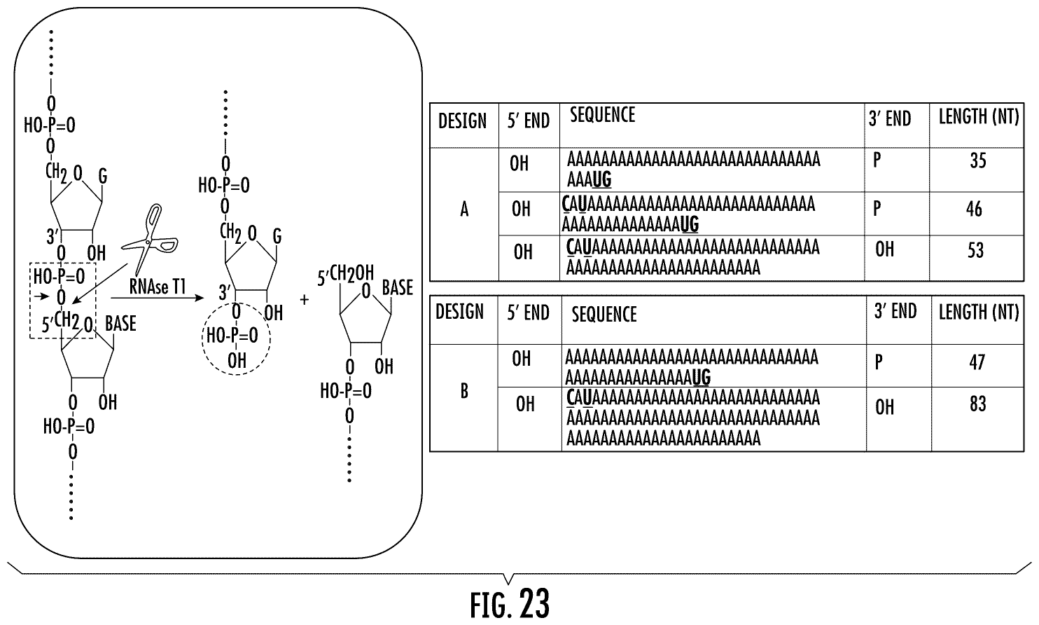

FIG. 23 (SEQ ID NOS: 12 to 16) depicts the cleavage site of the RNase T1 enzyme after each guanidine residue (left side) and the resulting poly(A) sequence fragments for mRNA samples (from EGFP mRNA) with design A and design B treated with RNase T1, and the expected 3′ functional groups to include a 3′ phosphate group where there is a guanidine cleavage or a 3′ hydroxyl group where there is no guanidine cleavage (right side). It was specifically observed that RNase T1 cleaves the phosphodiester bond between the 3′-guanylic residue and the 5′-OH residue of adjacent nucleotides, thus generating two (design A) or three (design B) fragments and the formation of corresponding intermediate 3′-cyclic phosphate (a phosphate group on the cleaved 3′ end of the poly(A) sample).

Being negatively charged, the resulting phosphate group on the cleaved 3′ end of the poly(A) sample was suspected to migrate earlier on a capillary electrophoresis system because it adds an additional charge in comparison to a hydroxyl group (—OH) in the size markers.

The standard treatment of mRNA samples must be kept consistent when cleaving poly(A) tails due to their fragmented design. In other words, cleavage does not occur just as one spot, but rather at two different guanidine residues so there needed to be size markers that have a 3′ phosphate at each of the size markers, and a hydroxyl group on the final size marker because there is no guanidine at the end of the poly(A) tail.

Accordingly, the 3′ end of the poly(A) size markers required optimization because it was contributing to a difference in size estimation of poly(A) tail length.

FIG. 24 (SEQ ID NOS: 17 to 31) depicts, for the purpose of comparison, an original set of size markers used to estimate the lengths of poly(A), each having a 3′ OH (upper). Round 1 shows an optimized set of size markers with length and base modifications and with either a 3′ P or a 3′ OH (lower).

The optimized size markers are commercially synthesized via chemical synthesis, a process of which is readily available in the field.

The original size markers were designed to be close in length to the expected length of poly(A) tail and (all of them having a 3′ hydroxyl group at the end) and compared to a set of optimized markers collected in Round 1. The Round 1 optimized set of size markers matched the base composition (in a different order to maintain proprietary sequence) and length of the expected poly(A) tails. Because RNase T1 generates a 3′ phosphate group upon cleavage after each guanidine residue on the mRNA poly(A) tail, optimized size markers were synthesized to more closely match the expected 3′ functional group of the cleaved mRNA samples. Duplicate Round 1 markers were synthesized to compare the migrations differences between markers ending with a 3′ phosphate and a 3′ hydroxyl group. Separation by capillary electrophoresis is affected by both size and charge. Modifying the base composition changes the size of the marker and the 3′ phosphate group adds additional charge to the molecule.

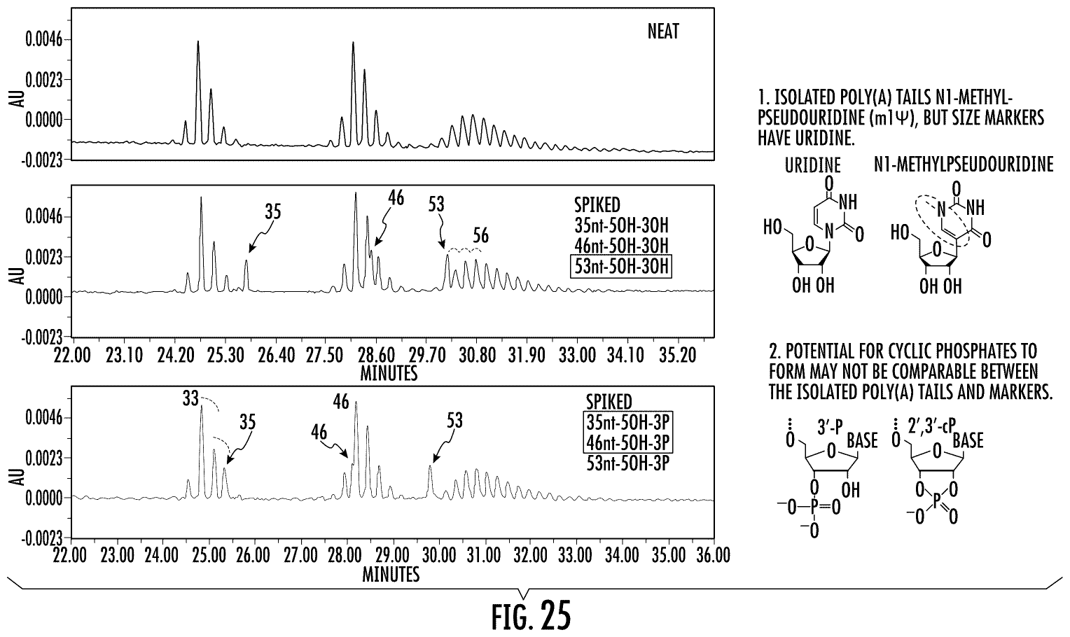

FIG. 25 depicts a series of electropherograms of isolated poly(A) tail cleaved from an mRNA sample (from EGFP mRNA) with design A illustrating a comparison of different functional groups on the 3′ end of the spiked size marker (OH or P) and the effect on migration (for example, at 35 nt) and peak splitting (for example, at 46 nt).

From top to bottom, the first panel shows the “neat” sample (isolated poly(A) tails without the marker spike-in) with different length distributions observed, and the size markers with (3′ OH, middle panel) and (3′ P, bottom panel) spiked-in to show the change in migration of the sequence caused by the 3′ functional groups. Obvious peak splitting is evident at the second panel and third panel at 46 nt.

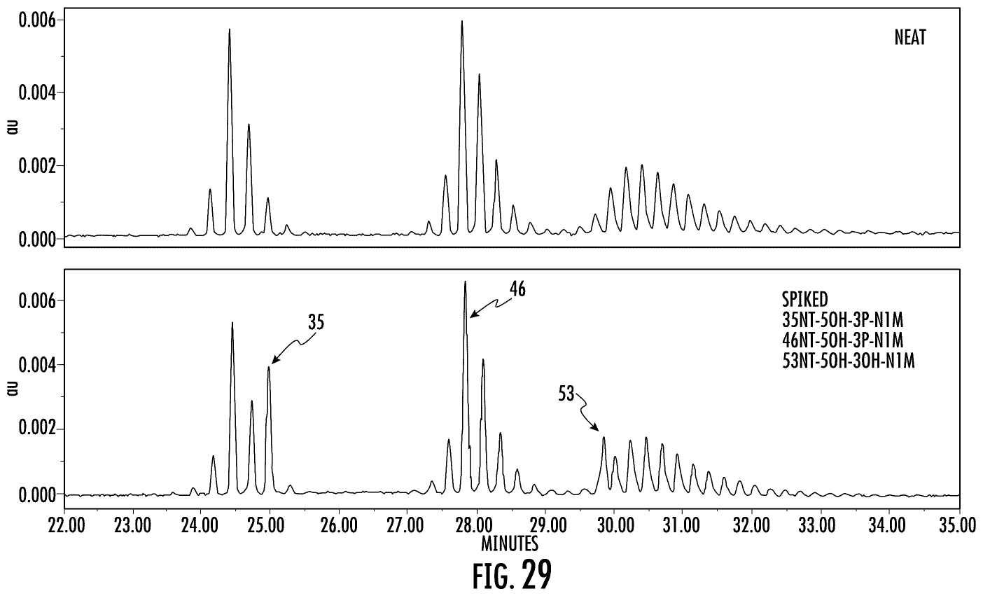

Two hypotheses were developed for the cause of peak-splitting. One, isolated poly(A) tails from the mRNA samples have a N1-methylpseudouridine (m1ψ or NIM) modified uridine. In other words, the size markers generated during Round 1 including a first set of optimized markers do not have an NIM modification. Two, on the phosphate generated end there is a potential for a cyclic phosphate to be generated and that could be migrating differently than the mRNA samples. Round 2 data of FIG. 29 supports the first hypothesis on the right side of FIG. 25, that is the presence of N1-methylpseudouridine.

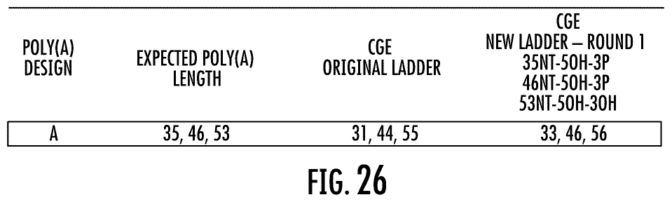

FIG. 26 depicts, for purpose of comparison, expected isolated poly(A) tail lengths under design A based on sequence, lengths determined using original ladder size markers (isolated poly(A) tails with the markers spiked-in) analyzed under design A, and optimized size markers selected for expected 3′ functional group based on the RNase T1 reaction (isolated poly(A) tails having a phosphate group or hydroxyl group on the 3′ end and with the marker spike-in) collected from the second and third panel of the electropherograms illustrated in FIG. 25.

In Round 1, the optimized size marker with the 3′ phosphate group was measured to have a length of 33 nt, 46 nt, and 56 nt. However, the expected poly(A) length was 35 nt, 46 nt, and 53 nt, respectively. To determine the appropriate size marker lengths, and determine whether the isolated poly(A) tails from the mRNA samples have a N1-methylpseudouridine (m1ψ or N1M) modified uridine, the mRNA samples were run through capillary gel electrophoresis mass spectrometry (CEMS) in Round 2 of testing.

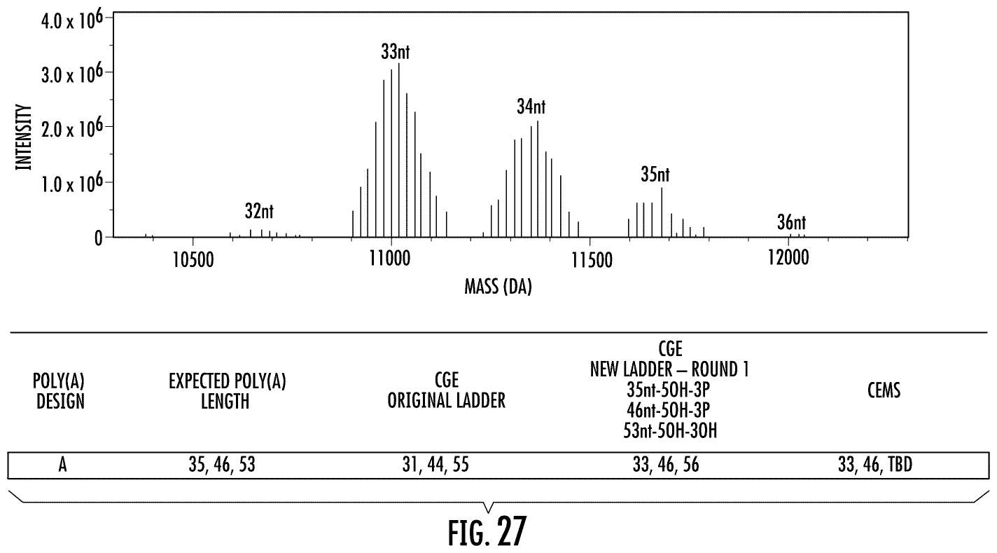

FIG. 27 depicts, for purpose of comparison, expected isolated poly(A) tail lengths under design A based on encoded sequence to generate mRNA samples, lengths determined using original ladder size markers (isolated poly(A) tails with the markers spike-in) analyzed under design A, and optimized size markers selected for expected 3′ functional group based on the RNase T1 reaction (isolated poly(A) tails having a phosphate group or hydroxyl group on the 3′ end and with the marker spiked-in) collected from the second and third panel of the electropherograms illustrated in FIG. 25, and the lengths assessed by capillary electrophoresis mass spectrometry (CE-MS).

The deconvoluted mass spectrum for the smallest fragment of design A is depicted in the graph above the table confirming the longest length to be 33 nt. However, the third distribution was a too low in intensity to determine a MS measurement.

FIG. 28 (SEQ ID NOS: 17 to 36) depicts, for the purpose of comparison, an original set of size markers used to estimate the lengths of poly(A) tail, each having a 3′ OH (upper). Round 1 shows an optimized set of size markers with length and base modifications and with either a 3′ P or a 3′ OH (lower). Round 2 shows a further optimized set of size markers with length and base modification with an N1-methyl-pseudouridine (m1ψ) modification on all of the uridines and a 3′ phosphate group or a 3′ hydroxyl group as expected for the RNase T1 enzyme reaction.

FIG. 29 depicts two electropherograms of isolated poly(A) tail analyzed under design A respectively illustrating the use of the further optimized size markers named ‘Round 2’ and the disappearance of the observed peak spitting in FIG. 25.

From top to bottom, the first panel shows the “neat” sample (isolated poly(A) tails without the markers spike-in) with different length distributions observed, and the second panel shows the optimized size marker with the N1-methyl-pseudouridine (m1ψ or N1M) modification on all of the uridines, optimized for the length and base composition, a 3′ phosphate (3′ P) for where there is a guanidine cleavage or 3′ hydroxyl (3′ OH) for where there is no guanidine cleavage

The results of Round 2 showed that when the new size markers are spiked-in, peak splitting on the spiked-in poly(A) samples is eliminated, therefore concluding that the isolated poly(A) tails from the mRNA samples have a N1-methyl-pseudouridine modified uridine. Separations using capillary electrophoresis are influenced by both size and charge. In this case the NIM modification affects the size of the cleaved poly(A) tail fragments. By matching the Round 2 optimized size markers with the same NIM modification, the cleaved poly(A) tail fragments migrated similarly on the capillary to more accurately determine the length of the poly(A) tail and remove the peak splitting.

RNase T1 enzyme is used to cleave the poly(A) tails from mRNA samples by selectively cleaving after guanidine residues, breaking the phosphodiester bond resulting in a phosphate group on the 3′ end. These cleaved poly(A) fragments are then analyzed by capillary electrophoresis (CE) where separation is based on differences in size and charge. Nucleotide bases (adenine, guanidine, uridine, and cytosine) have different sizes and were shown to affect the migration of the size markers spiked into the cleaved poly(A) samples in the CE separation.

A 3′ phosphate group adds an additional negative charge to the molecule as compared to a 3′ hydroxyl group and also affects the migration in a CE capillary. There were differences in the migration and size estimate between the original size markers and the Round 1 optimized size markers due to the differences in base composition and 3′ functional group.

Peak splitting was observed with the Round 1 spiked samples leading to a further optimization of the size marker as the uridines in the mRNA are N1-methyl-pseudouridine (m1ψ or N1M) modified, adding to the size of the cleaved poly(A) samples, and the Round 1 size markers were not.

Round 2 size markers were synthesized with the NIM modified uridines and the peak splitting disappeared. In summary, CE separations are influenced by size and charge and to use synthesized size markers to estimate the length of the cleaved poly(A) samples, they must match the base composition, any base modifications and 3′ functional group after RNase T1 cleavage.

While in the foregoing specification the inventions have been described in relation to certain aspects thereof, and many details have been put forth for the purpose of illustration, it will be apparent to those skilled in the art that the inventions are susceptible to additional aspects and that certain of the details described herein can be varied considerably without departing from the basic principles of the inventions.

1. A method for characterizing a polyadenylated region on a messenger ribonucleic acid (mRNA) that further comprises a coding region, wherein the method comprises the steps of:
(a) adjusting mRNA concentration in an mRNA sample based upon mRNA length;
(b) treating the mRNA sample with RNase T1 to remove the coding region and allow the polyadenylated region to remain;
(c) incubating the RNase T1 treated sample with oligo-dT beads;
(d) eluting the polyadenylated region bound to the oligo-dT beads;
(e) removing residual salts from the elution of step (d); and
(f) analyzing the polyadenylated region of step (e) using capillary gel electrophoresis (CGE).

2. The method according to claim 1, wherein step (d) uses nuclease free water.

3. The method according to claim 1, wherein step (e) uses a Zeba spin, dialysis, tangential flow flotation, or CAT cartridges.

4.-6. (canceled)

7. The method according to claim 1, further comprising, prior to step (f), mixing the mRNA sample with a population of mRNA ladders, wherein each mRNA ladder comprises at least one poly(A) sequence member having a guanine nucleotide spacer, wherein the mRNA ladder can have a nucleotide length of 12 to 250 nucleotides.

8.-13. (canceled)

14. The method according to claim 7, wherein an mRNA ladder comprises a poly(A) sequence member having 10 to 25 adenine residues with a guanine nucleotide spacer, and wherein the mRNA ladder has a hydroxyl group at the 3′ end.

15. The method according to claim 14, wherein the mRNA ladder further comprises a plurality of poly(A) sequence members having 10 to 25 adenine residues with a guanine nucleotide spacer.

16. The method according to claim 7, wherein the population of mRNA ladders comprises (i) a first mRNA ladder comprising at least one poly(A) sequence member having 10 to 25 adenine residues with a guanine nucleotide spacer, and wherein the first mRNA ladder has a hydroxyl group at the 3′ end, and (ii) a second mRNA ladder comprising at least one poly(A) sequence member having 10 to 25 adenine residues with a guanine nucleotide spacer, and wherein the second mRNA ladder has a phosphate group at the 3′ end.

17. The method according to claim 16, wherein the first mRNA ladder and the second mRNA ladder each further comprise at least one N1-methyl-pseudouridine nucleotide.

18. A characterized sample of a polyadenylated region of a messenger RNA (mRNA) that further comprises a coding region, wherein the characterized sample is produced according to the steps of:
a) adjusting mRNA concentration in an mRNA sample based upon mRNA length;
(b) treating the mRNA sample with RNase T1 to remove the coding region and allow the polyadenylated region to remain;
(c) incubating the RNase T1 treated sample with oligo-dT beads;
(d) eluting the polyadenylated region bound to the oligo-dT beads;
(e) removing residual salts from the elution of step (d); and
(f) analyzing the polyadenylated region of step (e) using capillary gel electrophoresis (CGE).

19. The characterized sample according to claim 18, wherein step (d) uses nuclease free water.

20. The characterized sample according to claim 18, wherein step (e) uses a Zeba spin, dialysis, tangential flow flotation, or CAT cartridges.

21.-23. (canceled)

24. The characterized sample according to claim 18, further comprising, prior to step (f), mixing the mRNA sample with a population of mRNA ladders, wherein each mRNA ladder comprises at least one poly(A) sequence member having a guanine nucleotide spacer, wherein the mRNA ladder can have a nucleotide length of 12 to 250 nucleotides.

25.-30. (canceled)

31. The characterized sample according to claim 24, wherein an mRNA ladder comprises a poly(A) sequence member having 10 to 25 adenine residues with a guanine nucleotide spacer, and wherein the mRNA ladder has a hydroxyl group at the 3′ end.

32. The characterized sample according to claim 31, wherein the mRNA ladder further comprises a plurality of poly(A) sequence members having 10 to 25 adenine residues with a guanine nucleotide spacer.

33. The characterized sample according to claim 24, wherein the population of mRNA ladders comprises (i) a first mRNA ladder comprising at least one poly(A) sequence member having 10 to 25 adenine residues with a guanine nucleotide spacer, and wherein the first mRNA ladder has a hydroxyl group at the 3′ end, and (ii) a second mRNA ladder comprising at least one poly(A) sequence member having 10 to 25 adenine residues with a guanine nucleotide spacer, and wherein the second mRNA ladder has a phosphate group at the 3′ end.

34. The characterized sample according to claim 33, wherein the first mRNA ladder and the second mRNA ladder each further comprise at least one N1-methyl-pseudouridine nucleotide.

35.-42. (canceled)

43. A population of messenger RNA ladders, wherein the population comprises:
(A) at least one messenger RNA ladder comprising (i) at least one poly(A) sequence member having 10 to 25 adenine nucleotides, (ii) at least one N1-methyl-pseudouridine nucleotide and (iii) at least one guanine nucleotide spacer, and wherein the mRNA ladder has a hydroxyl group at the 3′ end; and
(B) at least one messenger RNA ladder comprising (i) at least one poly(A) sequence member having 10 to 25 adenine residues, (ii) at least one N1-methyl-pseudouridine nucleotide and (iii) a guanine nucleotide spacer, and wherein the mRNA ladder has a phosphate group at the 3′ end.

44. The population of messenger RNA ladders, according to claim 43, wherein the poly(A) sequence members have 18 to 22 adenine nucleotides.

45. The population of messenger RNA ladders, according to claim 43, wherein the messenger RNA ladders of at least one of (A) or (B) further comprise at least one selected from the group consisting of a cytosine nucleotide and a uracil nucleotide.

46. The population of messenger RNA ladders, according to claim 43, wherein the messenger RNA ladders of at least one of (A) or (B) can have a nucleotide length of 12 to 250 nucleotides.

47. The population of messenger RNA ladders, according to claim 43, wherein the messenger RNA ladders of at least one of (A) or (B) can have a nucleotide length of 20 to 130 nucleotides.
